(19) United States
(12) Patent Application Publication
(10) Pub. No.: US 2022/0002809 A1
(43) Pub. Date: Jan. 6, 2022

(54) DNA METHYLATION BASED ESTIMATOR OF TELOMERE LENGTH

(71) Applicant: The Regents of the University of California, Oakland, CA (US)

(72) Inventors: Stefan Horvath, Los Angeles, CA (US); Ake Tzu-Hui Lu, Los Angeles, CA (US)

(73) Assignee: The Regents of the University of California, Oakland, CA (US)

(21) Appl. No.: 17/419,641

(22) PCT Filed: Feb. 5, 2020
(86) PCT No.: PCT/US20/16814

§ 371 Date: Jun. 29, 2021

Related U.S. Application Data
(60) Provisional application No. 62/801,797, filed on Feb. 6, 2019.

(51) Int. Cl. C12Q 1/6883; C12Q 1/6881

(52) U.S. Cl. CPC C12Q 1/6883; C12Q 2600/154; C12Q 1/6881

(57) ABSTRACT

The invention disclosed herein is a DNA methylation-based estimator of human telomere length (“DNAmTL”) that is based on 140 CpGs, and is applicable across the entire age spectrum. DNAmTL is even more strongly associated with chronological age than is measured TL (r˜−0.75 for DNAmTL versus r˜−0.35 for TL) and outperforms the latter in predicting i) time-to-death (P=4.1E-15), ii) time-to-coronary heart disease (p=6.6E-5), and iii) time-to-congestive heart failure (p=3.5E-6); all of which were corroborated with large sets of blood methylation data (N=6,850). DNAmTL is also associated with non-pathological conditions including physical functioning (P=7.6E-3), age-at-menopause (P=0.039), dietary variables (omega 3, fish, vegetable), educational attainment (P=4.3E-6) and income (P=3.1E-5). DNAmTL is an attractive molecular biomarker of aging due to its superior performance to measured TL, its intuitive interpretation of telomere length, its ease of use in vivo and in vitro, and its robustness.

### CROSS REFERENCE TO RELATED APPLICATIONS

This application claims the benefit under 35 U.S.C. Section 119(e) of co-pending and commonly-assigned U.S. Provisional Patent Application Ser. No. 62/801,797, filed on Feb. 6, 2019 and entitled “DNA METHYLATION BASED ESTIMATOR OF TELOMERE LENGTH” which application is incorporated by reference herein.

### STATEMENT OF GOVERNMENT INTEREST

This invention was made with government support under Grant Numbers AG051425 and AG060908, awarded by the National Institutes of Health. The government has certain rights in the invention.

### TECHNICAL FIELD

The invention relates to methods and materials for examining telomere length (TL) in individuals.

### BACKGROUND OF THE INVENTION

Telomeres are repetitive nucleotide sequences at the end of chromosomes that are not replicated during DNA synthesis of somatic cells. Therefore, telomeres shorten in function of each cell division. It is proposed that since the accumulated number of cell division in a body increases with age, telomere length can be used as a convenient age-indicator. Indeed, correlations between leukocyte telomere length (LTL) and chronological age can be as high as r=−0.51 for women and r=−0.55 for men. Although telomere lengths vary between tissues of the same body, their rate of attrition in blood, skin, muscle and fat tissues in the same adult is similar, suggesting that relative LTL can be an indirect indicator of the general state of health of the body. In this regard, it is more than note-worthy that shorter telomeres are associated with several conditions including cardiovascular disease, psychological stress, and lifespan. Another DNA based biomarker that changes with age is DNA methylation; specifically of cytosine residues of cytosine-phosphate-guanine dinucleotides (CpGs). Machine learning-based analyses of these changes generated algorithms, known as epigenetic clocks that use specific CpG methylation levels to accurately estimate age that is referred to as DNA methylation age (DNAm age). Although both DNAm age and LTL are associated with chronological age, they exhibit only weak correlations with each other. Several lines of evidence suggest that DNAm aging and telomere-associated aging are distinct mechanisms, e.g. age adjusted measures of DNAm age and LTL exhibit only weak correlations.

DNAm aging assays are already highly robust and ready for biomarker development. By contrast, the measurement of telomere length continues to encounter technical challenges and can be subject to technical confounding factors including but not limited to, the method used to extract DNA for telomere measurement. In view of this, there is a need for improved methods of observing telomere length and associated physiological characteristics.

### SUMMARY OF THE INVENTION

The invention disclosed herein provides a novel DNAm based biomarker, whose name, “DNAmTL”, reflects the fact that it is defined as surrogate biomarker of telomere length. As discussed below, DNAmTL has a variety of applications in epidemiological research and clinical settings. For example, DNAmTL can be used to complement existing clinical biomarkers when it comes to evaluating anti-aging interventions in vivo and in vitro. As DNAm captures important properties of the DNA molecule, these DNAm biomarkers are proximal to innate aging processes. In addition, DNAmTL can be used to enhance existing conventional lifespan predictors. Moreover, the surprising observation that DNAmTL outperforms conventionally observed LTL when it comes to lifespan prediction provides evidence that it is a superior biomarker than LTL (mean terminal restriction fragment “TRF”) when it comes to assessing the physiological state or mortality/morbidity risk of an individual. An age-adjusted version of DNAmTL is denoted as “DNAmTLadjAge” in the following text.

Beyond lifespan prediction, DNAmTLadjAge methodologies relate to most age-related conditions (metabolic syndrome, comorbidity, markers of dyslipidemia such as triglyceride levels) in the expected way, e.g. lower values of DNAmTLadjAge (corresponding to shorter telomeres) are associated with higher triglyceride. In addition, lower values of DNAmTLadjAge are associated with a blood cell composition that is indicative of older individuals and immunosenescence.

The invention disclosed herein provides DNA methylation based surrogate biomarkers of mean TRF, a measure that is widely used in physiological and epidemiological studies. In typical embodiments of the invention, artisans extract DNA from cells or fluids, e.g. human blood cells, whole blood, peripheral blood mononuclear cells, saliva, keratinocytes. Next, artisans can measure DNA methylation levels in the underlying signature of 140 CpGs (epigenetic markers) that are being used in the method. Typically, algorithms can provide estimates of LTL levels, more precisely mean TRF, for each sample or human individual. The higher the value, the lower the risk of death and disease. This DNAm based biomarker lends itself for imputing LTL on the basis of cytosine DNA methylation levels genomic locations (known as CpGs). To demonstrate that DNAmTL outperforms conventional observations of LTL, we carried out a large-scale meta analysis (over 1 k samples from the same individuals). We also characterized the resulting DNAmTL estimate with respect to lifestyle factors and a host of age-related conditions, e.g. we demonstrate that our DNAmTL predicts time to cardiovascular disease. As shown below, the disclosure presented herein demonstrates that DNAmTL is useful to monitor and evaluate interventions applied to age-related conditions.

Embodiments of the invention include, for example, methods of obtaining information on mean telomere length in an individual (e.g. so as to identify/characterize mean telomere length in the individual). Such methods typically include observing methylation of methylation markers present in SEQ ID NO.:1-SEQ ID NO.: 140 in genomic DNA from the individual; and then correlating methylation observed with mean telomere length such that information on mean telomere length in the individual is obtained. In illustrative embodiments of the invention, the genomic DNA used in the method is obtained from human leukocytes, fibroblasts, keratinocytes, buccal cells, endothelial cells, lymphoblastoid cells, and/or cells obtained from human blood, skin or saliva.

Embodiments of the invention also include methods of observing effects of a test agent on genomic methylation associated epigenetic aging of human cells. Typically these methods comprise combining the test agent with human cells, and then observing methylation in methylation markers of SEQ ID NO.:1-SEQ ID NO.: 140 in genomic DNA of the human cells exposed to the test agent. These methods then further include comparing the observations from these cells with observations of the methylation status in genomic DNA from control human cells not exposed to the test agent such that effects of the test agent on genomic methylation associated epigenetic aging in the human cells are observed.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIGS. 1A-1D provide graphed data showing measured LTL versus DNAmTL in training and test datasets. These graphs provide scatter plots of DNA methylation based telomere length (DNAmTL, x-axis) versus observed LTL measured by terminal restriction fragmentation (y-axis). DNAmTL and LTL are in units of kilobase. FIG. 1(A) shows graphed training data. FIG. 1(B) shows graphed data from the Framingham Heart Study. FIG. 1(C) shows graphed test data from the Women's Health Initiative (BA23 sub-study). FIG. 1(D) shows graphed test data from the Jackson Heart Study. Each panel reports a Pearson correlation coefficient and correlation test p-value.

FIGS. 2A-2F provide graphed data showing chronological age versus measured LTL and DNAmTL. These graphs provide data from studies on chronological age versus measured LTL (FIGS. 2A,2C,2E) and DNAmTL (FIGS. 2B,2D,2F). FIGS. 2A and 2B shows graphed test data from the FHS. FIGS. 2C and 2D show graphed test data from the WHI (N=100), FIGS. 2E and 2F show graphed test data from the JHS (N=100). Each panel reports a Pearson correlation coefficient and correlation test p-value.

FIGS. 3A-3B provide graphed data showing application of DNAmTL on in vitro keratinocytes, neonatal fibroblasts and adult coronary artery endothelial cells. FIG. 3(A) provides data depicting DNAmTL of keratinocytes from five heathy donors (y-axis, in units in kilobase) versus cumulative population doubling (x-axis). FIG. 3(B) provides data depicting DNAmTL of neonatal fibroblasts and adult coronary artery endothelial cells (EC), (y-axis, in units in kilobase) versus cumulative population doubling (x-axis). Both plots show that DNAmTL can track telomere attrition in non-leukocyte cells as a function of cumulative population doubling.

FIGS. 4A-4B provide graphed data showing application of DNAmTL on hTERT-transduced cells. FIG. 4(A) provides data showing DNAmTL tracking of telomere length of primary neonatal dermal fibroblasts from two donors, which were either un-transduced (Donor A and Donor B) or transduced immediately after their isolation (Donor A hTERT and Donor B hTERT). The telomeres of the neonatal fibroblasts, as measured by DNA TL (y-axis), underwent attrition as a function of cumulative population doubling (x-axis). FIG. 4(B) provides data showing DNAmTL tracking of telomere length of neonatal dermal fibroblast transduced with hTERT at different three points: OPD (the earliest time point with 0 population doubling and marked in red color), after 30PD (marked in pale red color with black borders), and after 40PD (marked in blood red color with black borders), respectively. Telomeres lengths of un-transduced control cells are represented by blue dots. DNAmTL detected the opposing activity of hTERT. Cells transduced with hTERT at the earliest time point (OPD) and hence have longest telomeres, shorten their telomeres even faster (slope=−0.01) than their un-transduced counterpart (marked in blue color, slope=−0.004). However, cells that were transduced with hTERT after 30 or 40 population doublings since their isolation, hence with shorter telomeres, experienced increased telomere lengths.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

All publications mentioned herein are incorporated herein by reference to disclose and describe aspects, methods and/or materials in connection with the cited publications. For example, U.S. Patent Publication 20150259742, U.S. patent application Ser. No. 15/025,185, titled “METHOD TO ESTIMATE THE AGE OF TISSUES AND CELL TYPES BASED ON EPIGENETIC MARKERS”, filed by Stefan Horvath; U.S. patent application Ser. No. 14/119,145, titled “METHOD TO ESTIMATE AGE OF INDIVIDUAL BASED ON EPIGENETIC MARKERS IN BIOLOGICAL SAMPLE”, filed by Eric Villain et al.; and Hannum et al. “GENOME-WIDE METHYLATION PROFILES REVEAL QUANTITATIVE VIEWS OF HUMAN AGING RATES” Molecular Cell. 2013; 49(2):359-367 and Lu et al., “DNA METHYLATION-BASED ESTIMATOR OF TELOMERE LENGTH” Aging (Albany N.Y.). 2019 Aug. 18; 11(16):5895-5923), are incorporated by reference in their entirety herein.

Novel molecular biomarkers of aging, such as those termed “DNAm age”, “epigenetic age” or “apparent methylomic aging rate” allow one to prognosticate mortality, are interesting to gerontologists (aging researchers), epidemiologists, medical professionals, and medical underwriters for life insurances. Exclusively clinical biomarkers such as lipid levels, body mass index, blood pressures have a long and successful history in the life insurance industry. By contrast, molecular biomarkers of aging have rarely been used.

The term “epigenetic” as used herein means relating to, being, or involving a chemical modification of the DNA molecule. Epigenetic factors include the addition or removal of a methyl group which results in changes of the DNA methylation levels.

The term “nucleic acids” as used herein may include any polymer or oligomer of pyrimidine and purine bases, preferably cytosine, thymine, and uracil, and adenine and guanine, respectively. The present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “methylation marker” as used herein refers to a CpG position that is potentially methylated. Methylation typically occurs in a CpG containing nucleic acid. The CpG containing nucleic acid may be present in, e.g., in a CpG island, a CpG doublet, a promoter, an intron, or an exon of gene. For instance, in the genetic regions provided herein the potential methylation sites encompass the promoter/enhancer regions of the indicated genes. Thus, the regions can begin upstream of a gene promoter and extend downstream into the transcribed region.

The phrase “selectively measuring” as used herein refers to methods wherein only a finite number of methylation marker or genes (comprising methylation markers) are measured rather than assaying essentially all potential methylation marker (or genes) in a genome. For example, in some aspects, “selectively measuring” methylation markers or genes comprising such markers can refer to measuring at least (or no more than) 25, 50, 75, 100 or 125 different methylation markers.

Novel “biomarkers of aging”, i.e. assessments that allow one to prognosticate mortality, are interesting to gerontologists (aging researchers), anti-aging researchers, epidemiologists, and medical professionals. The invention disclosed herein provides a novel DNAm based biomarker, whose name, DNAmTL, reflects the fact that it was defined as surrogate biomarker of leukocyte telomere length. As discussed below, DNAmTL has a variety of interesting applications in epidemiological research and clinical applications. DNAmTL provides useful biomarkers for human anti-aging studies given that it is a highly robust, blood based biomarkers that captures the physiological state, thus allowing efficacy of interventions to be evaluated based on real-time measures of aging, rather than relying on long-term outcomes, such as morbidity and mortality. In addition, this measure may be another component of the personalized medicine paradigm, as it allows for evaluation of risk based on an individual's personalized DNAm profile.

The conventional quantification of telomere length by terminal restriction fragment (TRF) is currently considered the gold standard in telomere biology (19). It assesses not only the telomeric region, but also the sub-telomeric region, which is variable, depending on the restriction enzyme that is used. Mean terminal restriction fragment (mean TRF) length is the highest standard of measurement of telomeres from total human genomic DNA. It is ideal if the robustness inherent in DNA methylation analyses is extended to telomere length measurement. Prior to the studies disclosed herein however, it was unknown whether one can indeed estimate mean TRF based on DNA methylation levels and if so, whether the resulting surrogate biomarker is biologically meaningful and useful.

We have developed a novel DNAm-based estimator of leukocyte telomere length, (DNAmTL), which estimates LTL based on DNA methylation levels of 140 CpGs that are found in Table 4. This epigenetic biomarker was developed by regressing LTL on blood methylation data from n=2,256 individuals (training set). We demonstrate that DNAmTL outperforms TRF-based LTL in predicting mortality and time-to-heart disease, as well as being associated with many age-related conditions, after adjusting for age. Beyond our comparative analysis, we also validate the utility of DNAmTL in a large-scale validation data set (N=6,850) for which LTL measurements were not available. Finally, our analyses also uncovered an association between age-adjusted DNAmTL with diet and clinical biomarkers. In this context, certain illustrative aspects and embodiments of the invention are published in Lu et al., “DNA METHYLATION-BASED ESTIMATOR OF TELOMERE LENGTH” Aging (Albany N.Y.). 2019 Aug. 18; 11(16):5895-5923), the contents of which are incorporate by reference.

Using an independent test set of n=1,078 samples, we demonstrate that DNAmTL is strongly correlated with actual LTL, as measure using TRF. However, a substantial deviation between DNAmTL and TRF-based LTL was observed in some individuals. This begs the question whether DNAmTL captures a biologically interesting aspect of telomere biology. Indeed, we find that the surrogate marker, DNAmTL outperforms the actual LTL (i.e. mean TRF) according to several criteria such as correlation with age, predictive power for time-to-death and time-to CHD/CHF. The large-scale meta-analysis (based on 6,800 Illumina arrays) confirms that age-adjusted DNAmTL (DNAmTLadjAge) predicts cardiovascular disease and lifespan. Unlike actual LTL, DNAmTL relates to physical functioning and age-at-menopause. The view that DNAmTLadjAge captures biologically interesting variation is also supported by our study of blood cell counts where DNAmTLadjAge is more strongly related to widely used biomarkers of immunosenescence (naive and exhausted cytotoxic T cells) than actual LTL.

Likewise DNAmTLadjAge also relates better to demographic characteristics (gender, race), lifestyle factors (diet, education, obesity), and several clinical biomarkers (lipid levels, insulin). Unlike LTL, DNAmTLadjAge is associated in the expected way to healthy diet (vegetable consumption, omega 3 intake) and obesity. Overall, these findings assert the superiority of DNAmTL over actual LTL when it comes to determining the effects of modifiable behavior.

We find that DNAmTLadjAge is associated with the four epigenetic clocks in the expected way, which is that low values of DNAmTLadjAge correspond to high epigenetic age acceleration. Comparative analysis of 3 large cohorts revealed that DNAmTL is in general inferior to epigenetic clocks (especially DNAm GrimAge (21)) when it comes to predicting lifespan and other age-related traits. This is unsurprising as some of the epigenetic clocks were trained using lifespan data. However, DNAmTL is superior to existing epigenetic clocks with respect to its simple biological interpretation (as alternative measure of telomere maintenance). Overall, our study demonstrates that DNAmTL is an attractive molecular biomarker of aging.

Technological platforms such as the Illumina Infinium microarray or DNA sequencing based methods have been found to lead to highly robust and reproducible measurements of the DNA methylation levels of a person. There are more than 28 million CpG loci in the human genome. Consequently, certain loci are given unique identifiers such as those found in the Illumina CpG loci database (see, e.g. Technical Note: Epigenetics, CpG Loci Identification ILLUMINA Inc. 2010). These CG locus designation identifiers are used herein. In this context, one embodiment of the invention is a method of obtaining information useful to observe biomarkers associated with telomere length in an individual by observing the methylation status of one or more of the 140 methylation marker specific GC loci that are identified in Table 4. Such sequences can further be characterized using one or more of the genomic databases that are readily available to artisans in this technology such as the UCSC Genome Browser, an on-line, and downloadable, genome browser hosted by the University of California, Santa Cruz (UCSC).

The invention disclosed herein has a number of embodiments. Embodiments of the invention include, for example, methods of obtaining information on mean telomere length in an individual (e.g. so as to identify/characterize mean telomere length in the individual). Such methods typically include observing methylation of methylation markers present in SEQ ID NO.:1-SEQ ID NO.: 140 in genomic DNA from the individual; and then correlating methylation observed with mean telomere length such that information on mean telomere length in the individual is obtained. In illustrative embodiments of the invention, the genomic DNA used in the method is obtained from human leukocytes, fibroblasts, keratinocytes, buccal cells, endothelial cells, lymphoblastoid cells, and/or cells obtained from human blood, skin or saliva.

In certain embodiments of the invention, methylation is observed by a process comprising treatment of genomic DNA from the population of cells with bisulfite to transform unmethylated cytosines of CpG dinucleotides in the genomic DNA to uracil; and/or methylation is observed by a process comprising hybridizing genomic DNA obtained from the individual with 140 complementary sequences disposed in an array and coupled to a substrate. In some embodiments of the invention, correlating methylation observed with mean telomere length comprises use of a weighted average of methylation markers and/or an algorithm such as use of a regression analysis. In addition, certain embodiments of the invention include using the observations (e.g. the specific methylation profiles) to estimate the phenotypic age of the individual. Optionally the methods comprise comparing the estimated phenotypic age with the actual age of the individual so as to obtain information on life expectancy or time-to-heart disease of the individual. Some embodiments of the invention further include using the observations to estimate the abundance of CD8+ T cells in the individual.

Other embodiments of the invention include methods of observing effects of a test agent on genomic methylation associated epigenetic aging of human cells. Typically these methods comprise combining the test agent with human cells (e.g. for at least one day, or at least one week, or at least one month), and then observing methylation in methylation markers of SEQ ID NO.:1-SEQ ID NO.: 140 in genomic DNA of the human cells. These methods then further include comparing the observations from these cells with observations of the methylation status in genomic DNA from control human cells not exposed to the test agent such that effects of the test agent on genomic methylation associated epigenetic aging in the human cells are observed. As is known in the art, typical control cell studies include essentially identical conditions to the studies with the test agent except that the control cells are not exposed to the test agent. In some embodiments of the invention, the test agent is a compound having a molecular weight less than 3,000 g/mol. In some embodiments of the invention, the test agent is a polypeptide or a polynucleotide. Optionally in these methods, a plurality of test agents are combined with the mammalian cells.

In the methods of observing effects of a test agent on genomic methylation associated epigenetic aging of human cells, genomic DNA used in these methods can be obtained from human leukocytes, fibroblasts, keratinocytes, buccal cells, endothelial cells, lymphoblastoid cells, and/or cells obtained from human blood, skin or saliva (i.e. these cell types are used in the methods). In some embodiments of the invention, genomic DNA used in the method is obtained from human cells of a leukocyte lineage, a neural cell lineage, a cardiac cell lineage or a skin cell lineage. In certain embodiments of the invention, these methods include comparing methylation profiles in cells from different tissue lineages (e.g. so as to compare methylation profiles in leukocyte, neural, cardiac, and skin cell etc. lineages).

In certain embodiments of these methods, methylation is observed using a weighted average of methylation markers; and/or methylation is observed using an algorithm such as a regression analysis. Optionally, methylation is observed by a process comprising treatment of genomic DNA from the population of cells from the mammals with bisulfite to transform unmethylated cytosines of CpG dinucleotides in the genomic DNA to uracil; and/or methylation is observed using a plurality of polynucleotides comprising the CpG sites coupled to a matrix such as a chip or a bead; and/or genomic DNA is amplified by a polymerase chain reaction process.

Yet another embodiment of the invention is a tangible computer-readable medium comprising computer-readable code that, when executed by a computer, causes the computer to perform operations comprising: receiving information corresponding to methylation levels of a set of methylation markers in a biological sample, wherein the set of methylation markers comprises methylation markers in SEQ ID NO.:1-SEQ ID NO.: 140; characterizing telomere length by applying an algorithm to methylation data obtained from the set of methylation markers; and then determining mean telomere length.

The invention disclosed herein provides a novel epigenetic biomarker that relates to telomere biology and telomere maintenance. In this context, it is critical to distinguish molecular biomarkers such as DNAmTL from clinical biomarkers of aging. Clinical biomarkers such as lipid levels, blood pressure, blood cell counts have a long and successful history in clinical practice. By contrast, molecular biomarkers of aging are rarely used. However, this is likely to change due to recent breakthroughs in DNA methylation based biomarkers of aging. DNA methylation (DNAm) based biomarkers of aging promise to greatly enhance biomedical research, clinical applications, patient care, and even medical underwriting. They will also be more useful for clinical trials and intervention assessment that target aging, since they are more proximal to the biological changes that characterize the aging process compared to upstream clinical read outs of health and disease status.

Our results surrounding the prediction of mortality and morbidity show that DNAmTL is highly robust and informative for a range of applications. While DNAmTL will probably not replace traditional biomarker assessments, it provides complementary information that adds valuable information, with potential clinical applications. DNAmTL can not only be used to directly predict/prognosticate mortality but also relate to a host of age-related conditions such as cardiovascular disease, cancer risk, and various measures of frailty.

As discussed below, we have developed a DNA methylation based surrogate biomarkers of mean TRF since the latter is widely used in physiological and epidemiological studies. The resulting DNAm based biomarker lends itself for imputing LTL on the basis of cytosine DNA methylation levels at 140 genomic locations (known as CpGs). To demonstrate that DNAmTL outperforms observed LTL we carried out a large-scale meta analysis (over 1 k samples from the same individuals). We also characterized the resulting DNAmTL estimate with respect to lifestyle factors and a host of age related conditions, e.g. we demonstrate that our DNAmTL predicts time to cardiovascular disease.

DNA methylation of the methylation markers (or markers close to them) can be measured using various approaches, which range from commercial array platforms (e.g. those made by Illumina™) to sequencing approaches of individual genes. This includes standard lab techniques or array platforms. A variety of methods for detecting methylation status or patterns have been described in, for example U.S. Pat. Nos. 6,214,556, 5,786,146, 6,017,704, 6,265,171, 6,200,756, 6,251,594, 5,912,147, 6,331,393, 6,605,432, and 6,300,071 and US Patent Application Publication Nos. 20030148327, 20030148326, 20030143606, 20030082609 and 20050009059, each of which are incorporated herein by reference. Other array-based methods of methylation analysis are disclosed in U.S. patent application Ser. No. 11/058,566. For a review of some methylation detection methods, see, Oakeley, E. J., Pharmacology & Therapeutics 84:389-400 (1999). Available methods include, but are not limited to: reverse-phase HPLC, thin-layer chromatography, SssI methyltransferases with incorporation of labeled methyl groups, the chloracetaldehyde reaction, differentially sensitive restriction enzymes, hydrazine or permanganate treatment (m5C is cleaved by permanganate treatment but not by hydrazine treatment), sodium bisulfite, combined bisulphate-restriction analysis, and methylation sensitive single nucleotide primer extension.

The methylation levels of a subset of the DNA methylation markers disclosed herein are assayed (e.g. using an Illumina™ DNA methylation array, or using a PCR protocol involving relevant primers). To quantify the methylation level, one can follow the standard protocol described by Illumina™ to calculate the beta value of methylation, which equals the fraction of methylated cytosines in that location. The invention can also be applied to any other approach for quantifying DNA methylation at locations near the genes as disclosed herein. DNA methylation can be quantified using many currently available assays which include, for example:

a) Molecular break light assay for DNA adenine methyltransferase activity is an assay that is based on the specificity of the restriction enzyme DpnI for fully methylated (adenine methylation) GATC sites in an oligonucleotide labeled with a fluorophore and quencher. The adenine methyltransferase methylates the oligonucleotide making it a substrate for DpnI. Cutting of the oligonucleotide by DpnI gives rise to a fluorescence increase.

b) Methylation-Specific Polymerase Chain Reaction (PCR) is based on a chemical reaction of sodium bisulfite with DNA that converts unmethylated cytosines of CpG dinucleotides to uracil or UpG, followed by traditional PCR. However, methylated cytosines will not be converted in this process, and thus primers are designed to overlap the CpG site of interest, which allows one to determine methylation status as methylated or unmethylated. The beta value can be calculated as the proportion of methylation.

c) Whole genome bisulfite sequencing, also known as BS-Seq, is a genome-wide analysis of DNA methylation. It is based on the sodium bisulfite conversion of genomic DNA, which is then sequencing on a Next-Generation Sequencing (NGS) platform. The sequences obtained are then re-aligned to the reference genome to determine methylation states of CpG dinucleotides based on mismatches resulting from the conversion of unmethylated cytosines into uracil.

d) The Hpall tiny fragment Enrichment by Ligation-mediated PCR (HELP) assay is based on restriction enzymes' differential ability to recognize and cleave methylated and unmethylated CpG DNA sites.

e) Methyl Sensitive Southern Blotting is similar to the HELP assay but uses Southern blotting techniques to probe gene-specific differences in methylation using restriction digests. This technique is used to evaluate local methylation near the binding site for the probe.

f) ChIP-on-chip assay is based on the ability of commercially prepared antibodies to bind to DNA methylation-associated proteins like MeCP2.

g) Restriction landmark genomic scanning is a complicated and now rarely-used assay is based upon restriction enzymes' differential recognition of methylated and unmethylated CpG sites. This assay is similar in concept to the HELP assay.

h) Methylated DNA immunoprecipitation (MeDIP) is analogous to chromatin immunoprecipitation. Immunoprecipitation is used to isolate methylated DNA fragments for input into DNA detection methods such as DNA microarrays (MeDIP-chip) or DNA sequencing (MeDIP-seq).

i) Pyrosequencing of bisulfite treated DNA is a sequencing of an amplicon made by a normal forward primer but a biotinylated reverse primer to PCR the gene of choice. The Pyrosequencer then analyses the sample by denaturing the DNA and adding one nucleotide at a time to the mix according to a sequence given by the user. If there is a mismatch, it is recorded and the percentage of DNA for which the mismatch is present is noted. This gives the user a percentage methylation per CpG island.

In certain embodiments of the invention, the genomic DNA is hybridized to a complimentary sequence (e.g. a synthetic polynucleotide sequence) that is coupled to a matrix (e.g. a bead or a chip). Optionally, the genomic DNA is transformed from its natural state via amplification by a polymerase chain reaction process. For example, prior to or concurrent with hybridization to an array, the sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159, 4,965,188, and 5,333,675. The sample may be amplified on the array. See, for example, U.S. Pat. No. 6,300,070, which is incorporated herein by reference.

In addition to using art accepted modeling techniques (e.g. convention algorithms such as regression analyses and the like), embodiments of the invention can include a variety of art accepted technical processes. For example, in certain embodiments of the invention, a bisulfite conversion process is performed so that cytosine residues in the genomic DNA are transformed to uracil, while 5-methylcytosine residues in the genomic DNA are not transformed to uracil. Kits for DNA bisulfite modification are commercially available from, for example, MethylEasy™ (Human Genetic Signatures™) and CpGenome™ Modification Kit (Chemicon™). See also, WO04096825A1, which describes bisulfite modification methods and Olek et al. Nuc. Acids Res. 24:5064-6 (1994), which discloses methods of performing bisulfite treatment and subsequent amplification. Bisulfite treatment allows the methylation status of cytosines to be detected by a variety of methods. For example, any method that may be used to detect a SNP may be used, for examples, see Syvanen, Nature Rev. Gen. 2:930-942 (2001). Methods such as single base extension (SBE) may be used or hybridization of sequence specific probes similar to allele specific hybridization methods. In another aspect the Molecular Inversion Probe (MIP) assay may be used.

The 140 CpG sites discussed herein are found Table 4. Table 4 includes DNA sequence information for each unique CpG locus cluster ID. Specifically, the Illumina method takes advantage of sequences flanking a CpG locus to generate a unique CpG locus cluster ID with a similar strategy as NCBI's refSNP IDs (rs #) in dbSNP (see, e.g. Technical Note: Epigenetics, CpG Loci Identification ILLUMINA Inc. 2010).

### Illustrative Aspects and Embodiments of the Invention

Telomeres are repetitive nucleotide sequences at the end of chromosomes that shorten with every cell division. Since the accumulated number of cell divisions increases with age, telomere length of proliferating cells exhibit negative correlations with age. Indeed, correlations between leukocyte telomere length (LTL) and chronological age can be as high as r=−0.51 for women and r=−0.55 for men (1). Although telomere length varies between tissues of the same body, their rate of attrition in blood, skin, muscle and fat tissues in the same adult is similar (2). Shorter telomeres are associated with cardiovascular disease, psychological stress, and lifespan (3-10). Another DNA-based biomarker that changes with age is DNA methylation; specifically of cytosine residues of cytosine-phosphate-guanine dinucleotides (CpGs). Machine learning-based analyses of these changes generated algorithms, known as epigenetic clocks that use specific CpG methylation levels to estimate chronological age (DNAm age) (11-14) and/or physiological age (15-17). Although both DNAm age and LTL are associated with chronological age, they exhibit only weak correlations with each other after adjusting for age (18-20), suggesting the distinct nature of their underlying mechanisms.

DNA methylation assays are already highly robust and ready for biomarker development; as reported by the BLUEPRINT consortium (21). By contrast, the measurement of telomere length continues to encounter technical challenges and can be subject to technical confounding factors including but not limited to even simple procedures such as the method used to extract DNA (22.23). Furthermore, the widely-used and highest standard of telomere measurement−mean terminal restriction fragment (mean TRF) assesses not only the telomeric region, but also the sub-telomeric region, which varies depending on the restriction enzyme that is used (22, 23). It would be ideal if the robustness inherent in DNA methylation analyses can be extended to telomere length measurement. It was however unknown whether one can indeed estimate telomere length based on DNA methylation levels and if so, whether the resulting epigenetic biomarker would be biologically meaningful and useful.

We present here, a novel DNAm-based estimator of leukocyte telomere length, (DNAmTL) that is based on DNA methylation levels of 140 CpGs. This epigenetic biomarker was developed by regressing measured LTL on blood methylation data from n=2,256 individuals (training set). We demonstrate that DNAmTL correlates negatively with age in numerous tissues and cell types, as would be expected of such a marker. Unexpectedly however, DNAmTL outperforms TRF-based LTL in predicting mortality and time-to-heart disease, as well as being associated with many age-related conditions, after adjusting for age. Beyond our training-test process, we also validated the applicability of DNAmTL on a large-scale data set (N=6,850) and uncovered associations between age-adjusted DNAmTL with diet and clinical biomarkers. In vitro studies demonstrated that DNAmTL is also applicable to cultured cells and correlates negatively with cell population doubling levels and tracks the dynamic changes of telomere length in response to hTERT expression; confirming that DNAmTL does indeed track telomere changes and not methylation changes that occur with cellular proliferation.

### Results

Training and Validation Data from 3 Cohorts

We evaluated data from n=3334 individuals for whom both LTL and Illumina methylation array data were available. These were from three different studies: Framingham Heart Study offspring cohort (FHS, N=878), Women's Health Initiative (WHI, N=818) and Jackson Heart Study cohort (JHS, N=1638, Table 1). The same laboratory measured LTL by Southern blotting of the terminal restriction fragments (TRFs)²⁴ and DNA methylation states by Illumina Infinium methylation array platform.

An overview of the data sets is found in Table 1. These US cohorts were comprised of two ethnic groups: 41% of European ancestry and 59% of African Ancestry. The age of the individuals ranged from 22 to 93 years. The training set used for constructing DNAmTL was comprised of N=2256 individuals from the WHI and JHS cohorts for whom LTL and DNAm data were assessed from the same blood sample (collected at the same time). Although fewer than 20% of individuals in the training set were of European ancestry, our test data demonstrate that the resulting DNAmTL estimator applies equally well to individuals of European ancestry. The test data set of N=1078 individuals was comprised of N=100 from the WHI, N=100 from JHS, and N=878 from the FHS cohorts. We used DNAmTL to interrogate other independent datasets from largescale cohort studies (N=6,850) for potential associations between telomere length and numerous age-related outcomes, conditions and lifestyle factors. We also further evaluated DNAmTL in publicly available data from adipose (N=648 from the Twins UK study (25, 26)), liver (N=85) (26, 27), and monocytes (n=1264 from the Multi-Ethnic Study of Atherosclerosis) (28). Finally, we tested DNAmTL in in vitro studies to ascertain its applicability to cultured cells and to probe the nature of DNAmTL's association with telomere length.

### DNAmTL Versus Measured TL in Blood and Adipose

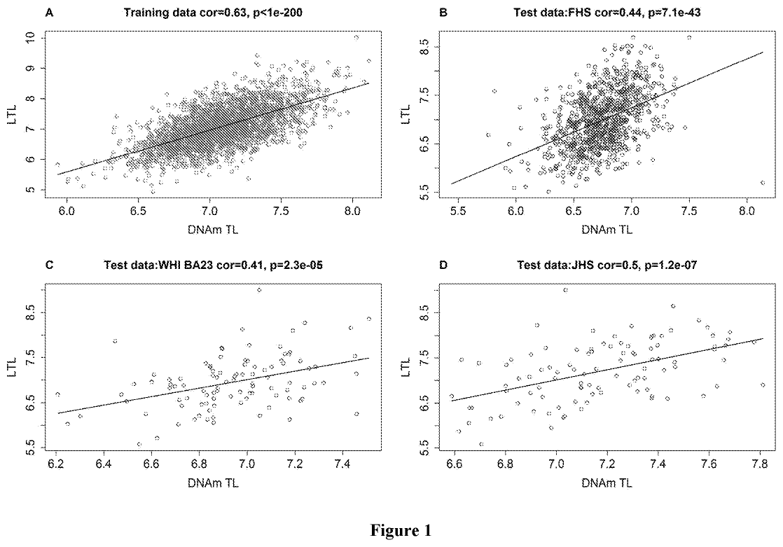

We focused the analysis on CpGs that are present on both the Illumina Infinium 450K array and the new Illumina EPIC methylation array in order to ensure future compatibility (Methods). Using the training data (n=2,256), we regressed measured LTL (mean TRFs) on blood CpG methylations using an elastic net regression model (29). This resulted in the automatic selection of 140 CpGs whose methylation levels best-predicted LTL. The linear regression model allows a direct prediction of TL based on DNA methylation levels. The predicted TL value, also referred to as DNAmTL, possesses the same units (kilobase) as that of mean TRF. The correlation coefficient between DNAmTL and LTL in the training data is r=0.63, which is overly optimistic, as independent validation with the test data sets produced correlations of r>0.40 (FIG. 1). It is noteworthy that with regards to the FHS cohort, LTL was measured in blood samples donated in exam 6, while the DNAm data were generated in blood samples donated 9.3 years later in exam 8, biasing the analysis toward the null hypothesis, i.e. the reported correlation (r=0.44, FIG. 1B) is overly conservative. This is supported by a separate analysis of non-blood tissue where a high correlation of r=0.65 between DNAmTL and TRF-based TL was revealed in adipose tissue samples from a Twins UK study.

DNAmTL Correlates More Strongly with Age than TL

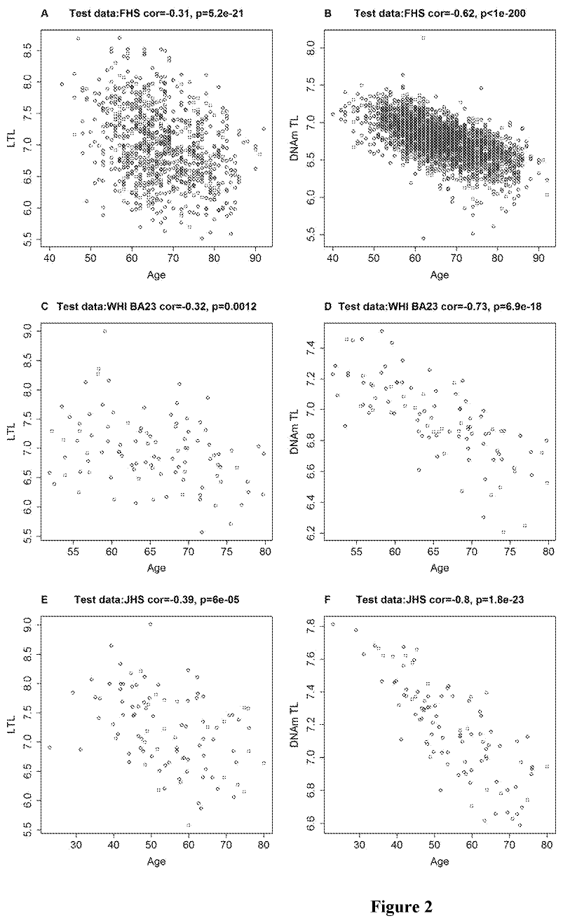

Although DNAmTL is an estimation of telomere length, it unexpectedly displays substantially stronger negative correlations with chronological age at the time of blood draw (r˜-0.80 to −0.62) than does measured LTL (r˜-0.40 to −0.30, FIG. 2). Multivariate regression models in the test data show that LTL shortens by 0.022 kilobases per year (P=2.3E-27) after adjusting for gender, race/ethnicity and other confounders (Table 2). Analogous multivariate regression models show that DNAmTL shortens by 0.018 kilobases per year, but this shortening is associated with a far more significant P value (P=6.0E-125) than that of measured LTL (P=2.3E-27). Although the DNAm-based biomarkers were derived from profiles of adults (22-93 years old), the resulting DNAmTL algorithm is equally applicable to profiles from children; even to those who are younger than 13 years of age, where a strong negative correlation of r=−0.71 was observed between DNAmTL of blood and chronological age. Such expected negative correlations with age were also seen with DNAmTL in adipose (r=−0.41), liver (r=−0.71), and in (sorted) monocytes (r=−0.60).

### Effect of Gender and Ethnicity

In addition to its correlation with age, TL has been repeatedly observed to exhibit significance differences between gender and ethnicity. For example, LTL of women are longer than that of men of the same age (30). The regression models we employed here also revealed that both LTL and its surrogate marker DNAmTL are indeed longer in females than in males. More importantly however, the p-values for LTL measured in this study (p=2.15E-4, N=1078) and that of a previous study (p=5E-3, N˜730) are far less significant than those for DNAmTL (p=1.14E-15, Table 2). We also find similarly longer DNAmTL in female liver samples compared to male liver samples of the same age (P=0.017). With regards to ethnicity, DNAmTL of PBMCs (Table 2) and monocytes revealed that US population of African ancestry have longer telomeres than those of European ancestry, echoing previous observations made with TRF-based measured telomere length). Once again, the correlation of DNAmTL (p=1.6E-33, N˜1200) with ethnicity was very much stronger than those seen with LTL measured by TRF (p=1E-4) or quantitative polymerase chain reaction (p=1E-3, N˜2450) (31). These strong correlations with age, gender and ethnicity confirms that DNAmTL is a reliable and robust surrogate as these are primary traits that have been previously established to be associated with telomere length.

### DNAm-Based TL is Often Superior to Measured LTL in Predicting Mortality and Health Outcomes

Having demonstrated the superior performance of DNAmTL over measured TL with regards to association with primary traits (age, gender and ethnicity), we sought to test whether it could reveal any correlations with health outcomes. Since chronological age would confound any potential relationship between DNAmTL and age-related traits such as health, it is useful to derive an age-adjusted estimate of DNAmTL (referred to as DNAmTLadjAge). Toward this end, we regressed DNAmTL on chronological age and the resulting raw residue was defined as DNAmTLadjAge. Thus, a negative value of DNAmTLadjAge indicates that the DNAmTL is shorter than expected based on chronological age, while a positive value would indicate the opposite. It is of interest to note that DNAmTLadjAge is heritable (heritability h²=0.46, P=4.5E-11) according to a pedigree-based polygenic model analysis in the FHS cohort (N>2000, Methods).

With this, we compared the performance of DNAmTLadjAge with that of age-adjusted measured LTL in the training and test datasets (N=3334) for which both measures were available. We find that longer DNAmTLadjAge is significantly associated with a lower hazard ratio (HR) for time-to-death, all-cause mortality (HR=0.31 and P=6.7E-9), time-to-coronary heart disease (HR=0.55 and CHD, P=9.5E-3), and time-to-congestive heart failure (HR=0.32 and CHF, P=9.7E-4). In women, later age at menopause was associated with significantly higher values of DNAmTLadjAge (P=0.025). Furthermore, physical activity was also positively associated with DNAmTLadjAge (P=0.013).

By comparison, the results from age-adjusted measured LTLs (LTLadjAge) were far less significant in predicting lifespan (P=4.7E-3 compared to P=6.7E-9 for DNAmTL) and were not significantly associated with time-to-CHD, time-to-CHF, age at menopause and physical activity (P>0.3).

These results provide evidence that DNAmTLadjAge outperforms LTLadjAge when it comes to predicting health-related outcomes. However, our comparative analysis was subject to a limitation: the measures of LTLadjAge and DNAmTLadjAge in the FHS cohort corresponded to two different blood samples collected at different time points. To address this limitation, we repeated the analysis by omitting the FHS data. In the resulting test data (n=100 samples from the WHI and n=100 samples from the JHS), DNAmTLadjAge continued to outperform LTLadjAge.

### Evaluating DNAmTL in Large Scale Validation Data

Having observed highly significant associations between DNAmTL with primary traits and numerous health outcomes using the training and test data sets, we sought to further validate these associations by analyzing even larger, independent data sets consisting of N=6,850 Illumina methylation arrays from blood samples of N=6,410 individuals from four cohorts: FHS, WHI, JHS and InChianti (Table 3, Methods). For most of these samples, measured LTL measurements were not available. The data set was comprised of three different ethnic groups: European (72%), African (17%), and Hispanic (11%) ancestries. The mean chronological age at the time of the blood draw was 65 years and the mean follow-up time (for all-cause mortality) was 12.3 years (Table 3). Once again, DNAmTL was negatively correlated with chronological age in these data sets (−0.83≤r≤−0.53).

Further analyses of these data confirmed that higher values of DNAmTLadjAge were indeed associated with longer lifespan. Each kilobase increase of DNAmTLadjAge was associated with a hazard ratio of 0.35 for mortality (P=4.1E-15), similar to what we observed in the training and test dataset (HR=0.31). Higher values of DNAmTLadjAge were also associated with longer time-to-CHD (HR=0.51 and P=6.6E-5) and longer time-to-CHF (HR=0.27 and P=3.6E-6), mirroring yet again the results obtained from the training and test data sets.

Two types of multivariate Cox regression models demonstrated that these associations remained significant even after adjusting for (1) blood cell counts, and (2) classical risk factors including body mass index, educational level, alcohol intake, smoking pack-years, prior history of diabetes, prior history of cancer, and hypertension status.

We went further and evaluated DNAmTLadjAge in different strata including age category (younger/older than 65 years), prevalent clinical conditions at baseline and found that DNAmTLadjAge remained a significant predictor of time-to-death in each of these strata, e.g. HR=0.26 for individuals aged<65 years and HR=0.41 for older individuals aged≥65 years. DNAmTLadjAge also remained a significant predictor of time-to-CHF in most strata and of time-to-CHD in specific strata such older age, normal BMI, or higher education attainment.

Our analyses revealed that higher DNAmTLadjAge values are associated with physical functioning (P=7.6E-3), and disease-free status (P=0.019), while prior history of cancer was associated with lower DNAmTLadjAge values (P=0.053). Interestingly, we also find that age-at-menopause is directly associated with DNAmTLadjAge (P=0.026). Our cross-sectional analyses however do not allow us to dissect cause-and-effect relationships, but it is nevertheless noteworthy that age-at-menopause is also associated with epigenetic ageing (32).

### DNAmTL Applied to In Vitro Cultured Cells

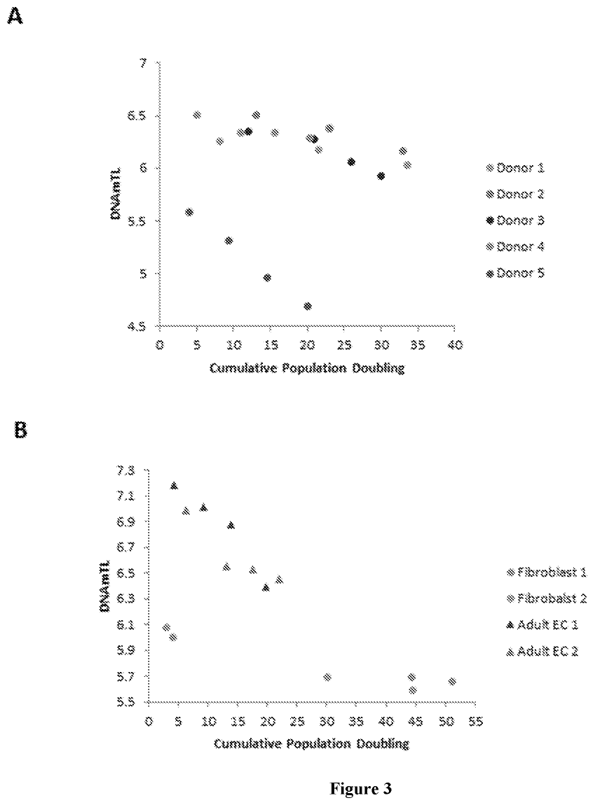

All the analyses above have thus far demonstrated that DNAmTL is a faithful surrogate not only for estimating telomere length but also for revealing known associations between TL and various primary biological traits (age, gender and ethnicity), as well as health outcomes, with far greater significance than was afforded by measured TL. DNAmTL goes even further by uncovering new associations with health traits that escaped linkage with measured TL. When a surrogate out-performs the intended target, it raises the obvious question of whether the former is truly tracking the latter, or whether the surrogate is tracking another feature that changes in parallel with the target. For the case in point, could DNAmTL instead, be tracking DNA methylation changes that occur simultaneously and precisely with cellular proliferation, just as telomere length does, but is nevertheless unrelated to telomere length? To obtain an insight into this question we proceeded to examine and characterize DNAmTL's performance with in vitro cultured cells, where much of telomere biology has been investigated and understood, and is a system that is far more amenable to experimentation. Primary keratinocytes isolated from healthy human skin were grown with serial passaging upon confluence, where cell numbers were obtained, and their population doublings determined. DNA methylation profiles of cells after different population doublings were measured using Illumina EPIC array and their telomere lengths estimated by DNAmTL. As would be expected, DNAmTL of keratinocytes from five heathy donors shortened in function of cumulative population doubling (FIG. 3). Interestingly, while the rates of telomere attrition of keratinocytes from neonatal donor 1 to 4 were mutually comparable, that of keratinocytes from adult donor 5 was markedly faster. Also DNAmTL of donor 5, who is 65 years old is considerably shorter than those of the neonatal donors, all of which are consistent with telomere biology.

Extending the application of DNAmTL to neonatal fibroblasts and adult coronary artery endothelial cells also revealed the expected association between telomere attrition and population doubling, as can be seen in FIG. 3B. Although, the adult coronary artery endothelial cells analyzed were from 19 and 26 year old donors, their telomeres are longer than those of neonatal fibroblasts, presumably reflecting tissue differences. What is consistent and clear however, is that their rates of telomere attrition are also greater than those of neonatal fibroblasts, echoing the telomere attrition rate of adult vs neonatal keratinocytes. The gradient of the curve (representing telomere attrition) of neonatal keratinocytes and fibroblasts ranged between −0.01 and −0.02, while that of adult keratinocytes and coronary artery endothelial cells ranged between −0.03 and −0.06. Collectively, these results demonstrate that DNAmTL exhibits the attributes expected of telomere length in regards to age of donor and association with cellular proliferation, and is hence applicable for in vitro cell culture studies.

DNAmTL Tracks Telomere Length in hTERT-Transduced Fibroblasts Cells

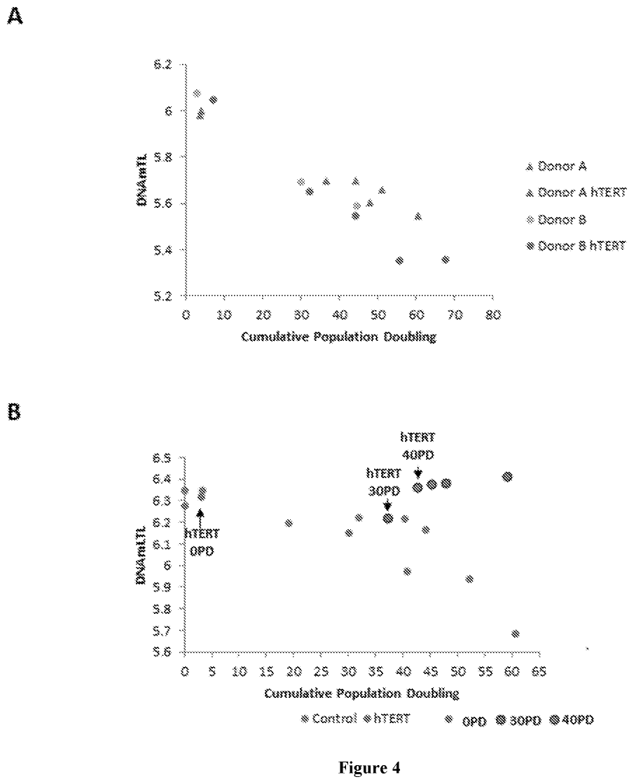

Having characterized DNAmTL's performance in cultured cells, we proceeded to determine whether DNAmTL does indeed track telomere length, or might it instead track DNA methylation changes that occur with cellular proliferation? Towards this end, we employed the hTERT protein, which is the catalytic subunit of the telomerase enzyme that can alter the length of telomeres. The ability of hTERT to replicate and elongate short telomeres is well-understood. Much less appreciated however, is the fact that hTERT actively shortens very long telomeres (33). This presented us with the opportunity to express hTERT in cells with telomeres of different lengths and see whether DNAmTL can detect and reveal the expected opposing outcomes. It would be a formidable benchmark for DNAmTL to achieve were it able to reveal such subtleties of hTERT action on telomeres, as it would confirm that it does indeed track telomere length and not cellular proliferation. First, we isolated primary dermal fibroblasts from two neonatal donors and transduced them with hTERT at the earliest time-point since their isolation, when their telomeres were the longest. As can be seen in FIG. 4A, the telomeres of all the neonatal fibroblasts, as measured by DNAmTL, decreased with cumulative population doubling and notably, those of hTERT-transduced cells were not elongated. Instead, they appeared to be even slightly shorter than their un-transduced counterparts. While this may seem surprising in the first instance, it is indeed consistent with hTERT's active shortening of very long telomeres, which these neonatal cells possess. To see if DNAmTL can detect the well-known telomere-lengthening activity of hTERT, we transduced neonatal dermal fibroblast at three different time-points from the time of their isolation. The earliest time-point of hTERT transduction was immediately following isolation when their telomeres were the longest (hTERT OPD for zero population doubling), while the second and third time-points, were after 30 population doublings (hTERT 30PD) and 40 population doublings (hTERT 40PD) respectively, when their telomeres would have shortened, as seen in FIG. 4A. The results imputed by DNAmTL are shown in FIG. 4B, where cells that were transduced with hTERT immediately after isolation (hTERT OPD) shortened their telomeres even faster (slope=−0.01 kilobase/PD) than their un-transduced counterpart (slope=−0.004 kilobase/PD), confirming the active trimming of long telomeres by hTERT. However, when hTERT was only introduced into cells after they have doubled 30 or 40 times, the lengths of their telomeres were increased, which is consistent with the well-documented elongation action of hTERT on short telomeres. The ability of DNAmTL to successfully detect opposing hTERT activities not only confirms the subtleties of the telomerase enzyme as previously reported (33), but more importantly, it proves that DNAmTL does indeed track telomere length and not cellular proliferation.

### Diet, Education, Life-Style Factors, Clinical Measurements and Biomarkers

To assess the effect of lifestyle factors and diet on DNAmTLadjAge in blood, we meta-analyzed large data sets from the FHS and WHI cohort (N up to 5,639, Methods) including their associations with clinical measurements. Age-adjusted DNAmTL was positively and clearly correlated with plasma-based estimates of mean carotenoid levels (robust correlation r=0.10, P=1.5E-5), beta-Cryptoxanthin (r=0.10 and P=3.8E-6) and high-density lipoprotein (HDL, r=0.04 and P=1.3E-3). Positive associations with DNAmTLadjAge were also observed for self-reported measures of vegetable (P=6.5E-4) fruit (P=0.027) and fish consumption (P=0.021). Positive correlations were also evident between DNAmTLadjAge and non-dietary life-style factors including level of educational attainment (P=4.3E-6), income (P=3.1E-5), and exercise (P=0.02). These remained true for each gender separately in the FHS cohort. There were also features that correlated negatively with DNAmTL. Smoking was associated with shorter DNAmTL in blood (r=−0.10, P=2.3E-14) and, incidentally, in adipose tissue as well (P=0.036). C-reactive protein (r=−0.09 and P=1.3E-8), triglyceride levels (r=−0.05 and P=4.3E-4) and insulin levels (P=3.5E-4) were negatively correlated with DNAmTLadjAge in both FHS and WHI cohorts. There were also negative correlations between DNAmTLadjAge (of blood) with waist-to-hip ratio (2.4E-4) and body-mass index (BMI, P=0.031).

Omega 3 Intake is Associated with Longer DNAmTL

The activities of omega-3 polyunsaturated fatty acid (PUFA) at the molecular and cellular level have led to projections that its supplementation could have a positive impact on health, especially on cardiovascular disease. Several large-scale studies, however, have not been able to provide an unambiguous conclusion, perhaps not surprisingly, since the nature of such studies are easily confounded by multiple uncontrollable factors, depending on the endpoint that is measured. Since CVD is also associated with telomere length, we employed DNAmTL to interrogate the potential effectiveness of PUFA. Interestingly, omega-3 intake was positively correlated with the age-adjusted DNAmTL (r=0.088 and P=4.4E-5) and to a considerably lesser extent, with age-adjusted TRF-based LTL (r=0.085 and P=0.016. For DNAmTLadjAge, the effect of omega 3 supplementation is more pronounced in males (r=0.08, P=0.012) than in females (r=0.047, P=0.11). A multivariate linear mixed effects model analysis confirmed the beneficial effect of omega-3 intake (suggestive P=0.09) even after adjusting for gender, BMI, educational levels, and smoking pack year.

### DNAmTL Relates to Imputed Blood Cell Composition

LTL is known to correlate with the abundance of naïve CD8+ T cells and other cell types (8). Similarly, we find DNAmTL significantly correlated with several quantitative measures of blood cells that were imputed using DNAm data (Methods, 33, 34) such as naïve CD8+ T cells (r=0.42, P=2.2E-151) and exhausted CD8+ T cells (r=−0.36, P=3.0E-102). A multivariate regression model revealed that 25% of the variation of age-adjusted DNAmTL and 4.6% of the variation in age-adjusted measured LTL can be attributed to imputed blood cell counts in the FHS test data. Overall, DNAmTL exhibits substantially stronger correlations with imputed blood cell counts than TRF-based LTL.

### Functional Annotation of CpGs Implicated in DNAmTL

Prior to this work, it was not immediately obvious that DNA methylation profiles can track telomere length. Although the numerous features (in vivo and in vitro) described above establish beyond doubt that DNAmTL can and does indeed do this, it remains unclear how this is achieved at the cellular and molecular level. To obtain a preliminary view into the link between DNA methylation and telomere length, we analyzed the genomic locations of the 140 CpGs underlying the DNAmTL using the GREAT software tool (36) which assigns potential biological meaning to a set of genomic locations (here CpGs) by analyzing the annotations of nearby genes. Ten gene sets were identified as significant at a stringent Bonferroni corrected significance level (P<0.05) including “Cadherin, N-terminal” that mediate cell adhesion, polarization and migration (nominal P=1.0E-7) and SODD/TNFR1 signaling pathway (nominal P=5.2E-5). When focusing only on the subset of 72 CpGs that have negative coefficients in the DNAmTL model (increasingly de-methylated with telomere shortening), we found 10 statistically significant gene sets at a Bonferroni corrected P<0.05 including calcium-dependent adhesion and “Cadherin, N-terminal” (P=2.8E-10). When focusing only on the 68 CpGs with positive coefficients in the DNAmTL model we identified 4 gene sets at a Bonferroni corrected P<0.05 including the vascular endothelial growth factor (VEGF) signaling pathway (P=7.4E-5). The expression of these gene sets remains to be validated, and for moment their identities do not immediately proffer obvious clues as to how they may be linked with telomere attrition, making their involvement all the more intriguing.

### DNAmTL is Often Inferior to Epigenetic Clocks

Numerous lines of evidence suggest that telomere attrition and epigenetic ageing are distinct cellular features that are well-associated with the ageing process. As such both telomere length and epigenetic clocks are potential estimators of biological age (9). To ascertain how related they are to each other, we calculated, pairwise correlations between DNAmTLadjAge and four “age-independent” epigenetic age acceleration measures derived from (1) the pan-tissue epigenetic clock by Horvath (2013)(37), (2) the blood-based clock by Hannum et al. (2013) (11), (3) the DNAm PhenoAge estimator by Levine et al. (2018) (15), and (4) the DNAm GrimAge estimator by Lu et al. (2019) (17). Using N=2356 samples from the FHS, we find that DNAmTLadjAge exhibits moderate negative correlations with the four epigenetic age acceleration measures (−0.44≤r≤−0.20).

We then compared the performance between DNAmTL and epigenetic clocks in predicting health outcomes. Using the validation data (N=6,850), we demonstrate that epigenetic age acceleration (AgeAccelGrim based on DNAm GrimAge) greatly outperforms DNAmTLadjAge with regards to predicting time-to-death (Cox P=5.8E-71 for AgeAccelGrim versus P=4.1E-15 for DNAmTLadjAge), time-to-CHD (P=2.5E-23 for AgeAccelGrim versus P=6.6E-5), and time-to-CHF (P=1.8E-35 versus P=3.6E-6).

The observation that telomeres shorten following each round of cellular replication was one of the most significant discovery in the field of ageing. In addition to explaining, many perplexing biological observations related to aging, it provided a potential means by which cellular ageing could be measured. In spite of the long passing of time since, this promise remains partially fulfilled. While there are admittedly many subtle biological factors that limit the speed of progress, there is in no small measure, technical challenges inherent in the process of measuring telomere length (38, 39). We have previously employed machine-learning methods to identify DNA methylation changes that occur with age and from these, generated highly-accurate epigenetic clocks that are very robust and compatible with different technological platforms. This robustness is due as much to the mathematical prowess of machine learning, as to the nature of the biomolecule that is measured, namely methylated DNA. Hence it is likely that measurement of telomere length would be more robust if it could also benefit from these two features. Less obvious however, is whether there is even a link between DNA methylation and telomere length that would allow such an approach to be successful. Notwithstanding this caveat, we proceeded with our attempt to derive a means to estimate telomere length based on methylation profile of the genome. To the best of our knowledge, this is the first study to describe a DNAm-based biomarker of leukocyte telomere length based on 140 CpGs. Using independent test data, we demonstrate that DNAmTL is strongly correlated with measured LTL (mean TRF) in blood and adipose tissue of individuals of different racial groups. Although the training data were blood DNA methylation profiles, and many of our epidemiological studies of DNAmTL involve blood methylation data, we show that DNAmTL is entirely applicable to other tissues as well (e.g. liver, adipose, and sorted monocytes). The general applicability of DNAmTL is important, if it were to be a powerful and robust tool. Similar accurate extrapolation of DNAmTL is seen with its applicability to the entire age-span, in spite of the fact that the training data were from adults (22-94 years old). The possible extension of DNAmTL to tissues and ages that were not in the training data sets indicates that it has successfully captured the underlying mechanisms that are related to telomere attrition.

However, a substantial deviation between DNAmTL and TRF-based LTL can be observed in some individuals. This begs the question whether DNAmTL captures a biologically meaningful aspect of telomere biology. Indeed, we were surprised to find that the surrogate marker, DNAmTL outperforms measured LTL (i.e. mean TRF) with regards to several criteria including correlation with age, predictive power for time-to-death and time-to CHD/CHF. Such a powerful and superior performance of a surrogate over the intended target raises many questions, as it appears at first sight to challenge logic and expectations. The most direct proposition is that although measured TL is subjected to technical challenges that limit its performance, it was still able to reveal the underlying principles associated with telomere attrition to machine learning processes, from which DNAmTL was successfully derived. Since DNAmTL is based on a bio-molecule (methylated CpG) that is much more accurately and robustly measured, it is perhaps un-surprising that its performance is likewise, more accurate and robust than mean TRF measurement. Indeed, this was the hope upon which this work was initiated. Consistent with this, DNAmTL is more strongly associated with traits already known to be linked with measured TL including demographic characteristics (gender, race), lifestyle factors (diet, education, obesity), and several clinical biomarkers (lipid levels, insulin). In addition to this, DNAmTL also revealed hitherto unknown associations between telomere length and features including physical functioning, age-at-menopause, healthy diet (vegetable consumption, omega 3 intake) and body mass index, in directions that are consistent with the known effects of these features on general good health. Overall, these findings assert the superiority of DNAmTL over measured LTL when it comes to determining the effects of modifiable behavior, which is clearly one of the most important features that will make DNAmTL a highly useful tool in seeking behavior and potential compounds that support healthy ageing. The view that DNAmTL captures biologically relevant variation is also supported by our study of blood cell counts where DNAmTLadjAge is more strongly related to widely used biomarkers of immunosenescence (naive and exhausted cytotoxic T cells) than are measured LTL.

Having outlined the strengths of the analyses, we wish to acknowledge several minor limitations and how they were mitigated. First, the training and validation data used in the development of DNAmTL differed in terms of the underlying racial composition. However, subsequent analyses demonstrated that DNAmTL applies to all groups—indicating yet again that DNAmTL has successfully captured the underlying principle associated with telomere length. Second, the DNA methylation data and the telomere measurements in the Framingham test data were collected at different time points as described above. In spite of this, the conclusions derived from those particular assessments were successfully confirmed with smaller data sets where both TL measurements and DNAmTL were carried out with the same DNA samples. Third, our training data focused only on blood samples, and while have indeed showed that DNAmTL also applies to adipose and liver tissue, we have only in vitro evidence that it also applies to keratinocytes, fibroblasts and endothelial cells. There is however, no reason to doubt that this in vitro observation would not hold true in vivo. These minor limitations notwithstanding, all in vivo and in vitro data are unanimous in demonstrating that DNAmTL provides not only a faithful estimation of telomere length, but is superior to measured TL in most, if not all circumstances.

The ability to extend the use of DNAmTL to in vitro studies was important not least because it allowed us to interrogate the possibility that DNAmTL does not actually track telomere length but instead tracks DNA methylation changes that occur in function of cellular proliferation. This is a distinct possibility because in such a hypothetical scenario, DNAmTL can still exhibit good correlation with measured TL even though they both track different features that change with cellular proliferation. This notion was however clearly discounted by in vitro experiments, where opposing effects of hTERT on telomere length were successfully detected by DNAmTL on cells that have undergone different number of population doublings, hence possess different telomere lengths. Expression of hTERT in cells with short telomeres resulted in greater DNAmTL than non-hTERT-expressing cells that have undergone similar or even more population doublings—a crucial observation that refutes the notion that DNAmTL might only be tracking cellular proliferation. In addition, DNAmTL successfully revealed and confirmed the lesser-known trimming activity of hTERT on long telomeres. Collectively, these in vitro observations provide unequivocal and direct support for the notion that DNAmTL does indeed directly track telomere length. What is far from clear however, and would require extensive investigation, is the bridge that connects telomere attrition with DNA methylation changes. In this regard, it was hoped that gene sets that are potentially implicated by virtue of their proximity to the 140 CpGs would provide some hints. While it is encouraging that genes with common themes were repeatedly identified, their known activities hitherto, do not automatically provide an intuitive link with telomere length or telomerase activity. This is all the more interesting as it provides evidence that hitherto unknown connections between cellular adhesion proteins, as well as cell signaling, and telomere attrition awaits discovery.

We initiated this investigation with the benefit of experience gained from our work with epigenetic clocks, (12, 14, 15, 17) which we have used in past investigations to demonstrate the distinctiveness between the process of telomere attrition and that of epigenetic ageing (40). This conclusion is corroborated by the results in this study. The epigenetic clocks and DNAmTL do not share CpGs and the respective genes proximal to their CpGs also do not overlap in function. While polycomb group protein targets feature prominently in epigenetic clock CpGs, they do not appear so in DNAmTL and conversely, cadherin and cell signaling genes that feature in DNAmTL do not do so in epigenetic clock CpGs. We find that DNAmTL is associated with the four epigenetic clocks in the expected way, which is that low values of DNAmTLadjAge correspond to high epigenetic age acceleration. Comparative analysis of 3 large cohorts revealed that DNAmTL is in general inferior to epigenetic clocks (especially DNAm GrimAge (17)) when it comes to predicting lifespan and other age-related traits. This is unsurprising as some of the epigenetic clocks were trained using lifespan data. However, DNAmTL is superior to existing epigenetic clocks with respect to its simple biological interpretation (as alternative measure of telomere length). Notwithstanding the clear difference between telomere attrition and epigenetic ageing, there is a moderate level of association between DNAmTL and epigenetic clocks, which is to be expected as they all (directly or indirectly) track age, in spite of their distinctiveness (41).

The successful transition of telomere length assessment into a DNA methylation-based assay allows DNA methylation levels that are profiled on a single DNA methylation platform, such as the Illumina DNA methylation array, to measure two distinct mechanisms of ageing—epigenetic ageing and telomere attrition, as well as other age-related health outcomes imputed by the other epigenetic clocks. This undoubtedly represents a step change in ageing research (in vivo and in vitro), as well as in health care and health management in the very near future.

Like epigenetic clocks, we expect that DNAmTL will become a useful biomarker in human interventional studies. A proof-of-concept study is provided by our preliminary analysis of omega-3 polyunsaturated fatty acid (PUFAs) supplementation. Several large-scale studies failed to detect convincing association between omega-3 PUFA supplementation and risk of cardiac death, sudden death, myocardial infarction, stroke, or all-cause mortality (42-44). However, we find omega-3 intake to be positively correlated with DNAmTLadjAge (r=0.088 and P=4.4E-5) and to a considerably lesser extent with age-adjusted TRF-based LTL (r=0.085 and P=0.016). While future randomized controlled trials should aim to validate these associations, these preliminary results show that blood-based epigenetic biomarkers readily lend themselves for detecting beneficial effects of a promising dietary supplement. Overall, we expect that DNAmTL will become an attractive molecular biomarker of aging due to its greater performance than measured TL, its intuitive interpretation as epigenetic biomarker of telomere maintenance, and its ease of use and robustness.

### Methods

### Epidemiological Cohorts

To establish DNAm-based telomere length in leukocytes we used N=2256 individuals from the WHI BA23 and JHS cohort in the training process and N=1078 individuals from the FHS cohort (45), WHI (46,47), JHS (48) cohort in the test process, as listed in Table 1.

Our validation analyses involved N=6,850 Illumina arrays measuring blood methylation levels in N=6,410 individuals from four independent cohorts: the FHS dataset (N=2,356), WHI BA23 (N=1,389), WHI EMPC (N=1972), JHS (N=209), and InChianti (N=924 from 1 to 2 longitudinal measures on 484 individuals, Table 3). All statistical analyses were adjusted for the correlation structure due to pedigree effects or repeated measurements as described below.

### LTL Measurements

The same lab generated the LTL data across the 3 cohorts (24). LTL was measured using Southern blots of the terminal restriction fragment length. After extraction, DNA was inspected for integrity, digested, resolved by gel electrophoresis, transferred to a membrane, hybridized with labeled probes and exposed to X-ray film using chemiluminescence, as previously described in (24). The inter-assay coefficient of variation for blinded pair sets was 2.0% for the WHI, 1.4% for the JHS and 2.4% for the FHS (24).

### Estimation of Surrogate DNAm Based Telomere Length in Leukocytes

We developed an estimate of LTL based only on DNA methylation levels. The estimate was established using the elastic net regression model implemented in the R package glmnet²⁹. The elastic net regression model corresponds to a choice of 0.5 for the alpha parameter in the glmnet function. Ten-fold cross validation was performed in the (WHI and JHS) training data to specify the underlying tuning parameter k. The final model was based on lambda.1se, i.e., the λ value that led to the minimum cross validated error within one standard error.

### DNA Methylation Data

The DNA methylation profiling was based on the Illumina Infinium HumanMethylation450K BeadChip in the FHS and WHI cohort and was based on the Illumina Infinium EPIC 850K BeadChip in the JHS cohort. To ensure future use with EPIC arrays, we focused on the subset of 450,161 CpGs that were present on both platforms. We kept the original normalization methods to ensure consistency with previous publications. The WHI BA23 were normalized using the background correction method implemented in the software GenomeStudio. The JHS data were normalized using the “noob” normalization method implemented in the minfi R package (49, 50).

### Statistical Models Used in Validation Analysis

Our validation analysis involved i) Cox regression for time to death (all-cause mortality), for time-to-CHD, for time to coronary heart failure, and time to any cancer, ii) linear regression for our DNAm based measures (independent variable) associated with and number of age-related conditions (dependent variable) and physical function score, respectively, iii) linear regression for age at menopause (independent variable) associated with our DNAm measure, iv) logistic regression analysis for estimating the odds ratios of our DNAm based measure associated with any cancer, hypertension, type 2 diabetes, and disease free status. The multimorbidity index was defined as the number of age-related conditions including arthritis, cataract, cancer, CHD, CHF, emphysema, glaucoma, lipid condition, osteoporosis, type 2 diabetes. In our validation analysis, we used the age independent variable, DNAmTLadjAge. All regression models included the following covariates: age, gender, and batch effect as needed. To avoid bias due to familial correlations from pedigrees in the FHS cohort or intra-subject correlations resulting from repeated measurements, we used the following techniques. For censored time variables, we used robust standard errors, the Huber sandwich estimator, implemented in R coxph function. We used linear mixed models with a random intercept term, implemented in lme R function. We used generalized estimation equation models (GEE), implemented in R gee function, for our logistic regression models. Additional covariates related to demographic characteristics, psychosocial behaviors and clinical covariates were adjusted in multivariate Cox models analysis. Those additional covariates includes BMI (category), education attainment (category), alcohol consumption (gram/day), self-reported smoking pack-years, three medical covariates: status of cancer, hypertension and type 2 diabetes at baseline. The categories associated with BMI ranges are a) 18.5-25 (normal), b) 25 to 30 (over), and c) >30 (obese). The categories associated with educational attainment are a) less than high school, b) high school degree, c) some college, and d) college degree and above. Smoking pack-years and educational variables were not available in the JHS cohort. Smoking category (never, former and current) was used in the analysis using the JHS cohort. Our stratified analysis was conducted in strata defined by age (<65 versus ≥65 years), BMI, education, prior history of hypertension, type 2 diabetes or cancer. All models used in the stratified analysis adjusted for age, gender, and (possibly) batch effect.

### Meta-Analysis

We typically used fixed effects meta-analysis models weighted by inverse variance to combine the results across validation study sets into a single estimate. Toward this end, we used the metafor R function. Alternatively, we used Stouffer's meta-analysis method (weighted by the square root of sample size) to combine results for variables whose scale/definition differed across study sets, e.g. multimorbidity (number of age-related conditions), disease free status and physical function scores.

Cox Models that Include Blood Cell Counts

We also fit multivariate Cox regression models that adjusted for imputed blood cell counts in addition to chronological age, batch, and pedigree structure, for predicting time-to-death and time-to-CHD. The blood cell counts were imputed based on DNA methylation levels as described elsewhere. To avoid multi-collinearities between blood cell counts, we only included the following seven blood cell counts into the multivariate model: naïve CD8+T, exhausted cytotoxic CD8+ T cells, plasma blasts, CD4+T, natural killer cells, monocytes and granulocytes.

### Heritability Analysis

In general, DNAm based biomarkers are highly heritable (19, 51, 52) To evaluate whether DNAmTLadjAge is heritable as well, we estimated the narrow sense heritability h² using the polygenic models defined in SOLAR (53) and its R interface solarius (54). Heritability is defined as the total proportion of phenotypic variance attributable to genetic variation in the polygenic model. The quantitative trait DNAmTL was adjusted for both age and gender. The robust polygenic model (with the option of a t-distribution) was used to estimate heritability. The heritability estimate correspondents to the variance component associated with the kinship coefficient. We used all individuals from the FHS cohort for whom DNA methylation data were available (irrespective of the availability of the observed LTL measure).

### LTL Measures Versus Blood Cell Composition

The imputed blood cell abundance measures were related to TRF based and DNAm based LTL measures, using the training datasets from the WHI BA23 and the JHS cohort and the test dataset from the FHS cohort, involving n=3,134 individuals. The following imputed blood cell counts were analyzed: B cell, naïve CD4+T, CD4+T, naïve CD8+T, CD8+T, exhausted cytotoxic CD8+ T cells (defined as CD8 positive CD28 negative CD45R negative), plasma blasts, natural killer cells, monocytes, and granulocytes. The blood cell composition imputation of the naive T cells, exhausted T cells, and plasma blasts was based on the Horvath method (55). The remaining cell types were imputed using the Houseman method (35). To avoid confounded by age, we used the age-adjusted DNAmTL (DNAmTLadjAge) variable for analysis. The correlation results were combined across studies via the same fixed effects meta-analysis model.

### GREAT Analysis

We applied the GREAT analysis software tool (36) to three sets of CpGs: (1) all the 140 CpGs underlying DNAmTL model, (2) the 72 CpGs with negative coefficients in the model, and (3) the other 68 CpGs with positive coefficients in the model. CpGs in non-coding regions typically lack annotation with respect to biological functions. GREAT assigns biological meaning to a set of non-coding genomic regions (implicated by the CpGs) by analyzing the annotations of the nearby genes. Toward this end, the GREAT software performs both a binomial test (over genomic regions) and a hypergeometric test over genes when using a whole genome background. We performed the enrichment based on default settings (Proximal: 5.0 kb upstream, 1.0 kb downstream, plus Distal: up to 1,000 kb) for gene sets associated with GO terms, MSigDB, PANTHER, KEGG and InterPro pathway. To avoid large numbers of multiple comparisons, we restricted the analysis to the gene sets with between 5 and 3,000 genes. We report nominal P values and two adjustments for multiple comparisons: Bonferroni correction and the Benjamini-Hochberg false discovery rate.

### Diet and Lifestyle Factors

We performed a robust correlation analysis (biweight midcorrelation, bicor (56)) between DNAmTLadjAge and 41 variables including 13 self-reported dietary variables, 9 dietary biomarkers, 14 variables related to metabolic related traits and central adiposity, and 5 life style factors, using the FHS and/or WHI cohort. In the FHS cohort, we conducted the robust correlation analysis in males and females, respectively. Next we combined the results via fixed effect models weighted by inverse variance. In the FHS cohort, we used linear mixed effects models to account for pedigree structure. In the WHI cohort, we conducted the robust correlation analysis in each ethnic group separately. Next we combined the results via fixed effects meta-analysis models. The final results were based on the meta-analysis that combined the results across the FHS and WHI cohorts using the fixed effect models weighted by inverse variance. In the FHS cohort, the diet and the clinical variables were based on the datasets archived in dbGAP pht002350.v4.p10 and pht000742.v5.p10, respectively. Both datasets were collected at exam 8, aligned with the blood drawn for DNA methylation profiles. In the WHI cohort, blood biomarkers were measured from fasting plasma collected at baseline. Food groups and nutrients are inclusive, including all types and all preparation methods, e.g. folic acid includes synthetic and natural, and dairy includes cheese and all types of milk. The individual variables are explained in (57).

### In Vitro Cultured Cell Procedure

### Isolation and Culture of Primary Cells

Primary human neonatal fibroblasts were isolated from circumcised foreskins. Informed consent was obtained prior to collection of human skin samples with approval from the Oxford Research Ethics Committee; reference 10/H0605/1. The tissue was cut into small pieces and digested overnight at 4° C. with 0.5 mg/ml Liberase DH in CnT-07 keratinocyte medium (CellnTech) supplemented with penicillin/streptomycin (Sigma) and gentamycin/amphotericin (Life Tech). Following digestion, the epidermis was peeled off from the tissue pieces. The de-epidermised tissue pieces were placed faced down on plastic cell culture plates and allowed partially dry before addition of DMEM supplemented with 10% FBS and antibiotics. After several days incubation in a 37° C., 5% CO₂ humidified environment, fibroblasts can be seen to migrate out from the tissue pieces and when their growth reached confluence, they were trypsinized, counted and seeded into fresh plates for experiments. Adult human coronary artery endothelial cells (HCAEC) were purchased from Cell Applications (USA) and cultured in MesoEndo Cell Growth Medium (Sigma) at 37° C. humidified incubator with 5% CO₂.

### Neonatal Foreskin Fibroblasts

100,000 cells were seeded into a 10 cm plate and cultured as described above. Upon confluence the cells were harvested with trypsin digestion followed by neutralisation with soybean trypsin inhibitor. The number of cells was ascertained and 100,000 was taken and seeded into a fresh plate. The remaining cells were used for DNA extraction. Population doubling was calculated with the following formula: [log(number of cells harvested)−log(number of cells seeded)]×3.32. Cumulative population doubling was obtained by addition of population doubling of each passage.

### Adult Human Coronary Artery Endothelial Cells

500,000 cells were seeded into a fibronectin-coated 75 cm² flask and cultured as described. The procedure of passing the cells, counting and ascertaining population doubling is similar to those described for neonatal foreskin fibroblasts above.

Retroviral-Mediated Transduction of Cells with hTERT Vectors

Retroviral vectors bearing wildtype hTERT (Addgene, cat. 1774), was transfected into Phoenix A cells using the calcium chloride method according to the manufacturer's instructions (Profection Cat No: E1200 Promega). They next day, media were removed from the transfectants and replaced with DMEM supplemented with 10% foetal calf serum. The following day, the media containing recombinant retroviruses were collected, filtered through 0.45 micron filter and mixed with polybrene (Sigma) up to 9 ug/ml and used to feed the cells intended for infection. The next day, fresh media containing puromycin (1 ug/ml) was given to the cells. After 3-4 days when all the control cells in the uninfected plates were dead, the surviving infectants were grown and used for experiments as described above.

### DNA Extraction in the In Vitro Experiments

DNA was extracted from cells using the Zymo Quick DNA mini-prep plus kit (D4069) according to the manufacturer's instructions and DNA methylation levels were measured on Illumina 850 EPIC arrays according to the manufacturer's instructions.

TABLES

TABLE 1
Overview of training and test data

| | | | | | | | Median |
|---|---|---|---|---|---|---|---|
| | | | | | Median | Median | years of |
| | | | Fe- | | LTL | Age | follow-up |
| Data | Type | N | male | Race | (range) | (range) | (25^{th}, 75^{th}) |
| WHI | Train | 718 | 100% | European | 6.9 | 66.5 | 18.9 |
| BA23 | | | | (59%) | (5.2, 9.1) | (50.2, | (17.4, 19.9) |
| | | | | AfricanA | | 80.2) | |
| | | | | (41%) | | | |
| JHS | Train | 1538 | 64% | AfricanA | 7.1 | 56.6 | 12.2 |
| | | | | (100%) | (4.9, 10) | (22.2, | (11.3, 13) |
| | | | | | | 93.1) | |
| FHS | Test | 878 | 51% | European | 7 | 57.0 | 8.2 |
| | | | | (100%) | (5.5, 8.7) | (33.0, | (7.4, 8.9) |
| | | | | | | 82.0) | |
| WHI | Test | 100 | 100% | European | 6.9 | 65.3 | 18.9 |
| BA23 | | | | (49%) | (5.6, 9) | (51.9, | (15.5, 19.9) |
| | | | | AfricanA | | 79.8) | |
| | | | | (51%) | | | |
| JHS | Test | 100 | 55% | AfricanA | 7.2 | 53.5 | 12.1 |
| | | | | (100%) | (5.6, 9) | (22.9, | (11.2, 12.9) |
| | | | | | | 80) | |

AfricanA = African American.

### Legend

Characteristics of study participants in the training and test data sets that were used to develop and validate DNAmTL.

TABLE 2
Multivariate regression analysis of leukocyte telomere length

| | | | |
|---|---|---|---|
| Variable | Coefficient (SE) | t-statistic | P |

Outcome: actual LTL (mean TRF)

| | | | | |
|---|---|---|---|---|
| Intercept | 8.43 | (0.201) | 41.88 | 2.43E−227 |
| Age | −0.022 | (0.002) | −11.14 | 2.33E−27 |
| Female | 0.132 | (0.036) | 3.71 | 2.15E−4 |
| Race: European | 0.029 | (0.112) | 0.26 | 7.97E−1 |
| smoke: Former | 0.132 | (0.063) | 2.11 | 3.50E−2 |
| smoke: Never | 0.113 | (0.062) | 1.82 | 6.95E−2 |
| BMI | −0.007 | (0.003) | −2.15 | 3.16E−2 |
| JHS | 0.005 | (0.126) | 0.04 | 9.66E−1 |
| WHI BA23 | −0.093 | (0.084) | −1.10 | 2.73E−1 |

Outcome: DNAmTL

| | | | | |
|---|---|---|---|---|
| Intercept | 8.046 | (0.069) | 116.16 | <2.0E−200 |
| Age | −0.018 | (0.001) | −27.32 | 5.97E−125 |
| Female | 0.099 | (0.012) | 8.14 | 1.14E−15 |
| Race: European | −0.136 | (0.039) | −3.52 | 4.57E−4 |
| smoke: Former | 0.08 | (0.022) | 3.72 | 2.09E−4 |
| smoke: Never | 0.096 | (0.021) | 4.51 | 7.11E−6 |
| BMI | −0.002 | (0.001) | −2.19 | 2.91E−2 |
| JHS | 0.069 | (0.044) | 1.59 | 1.11E−1 |
| WHI BA23 | 0.049 | (0.029) | 1.69 | 9.15E−2 |

BMI = body mass index; SE = standard error.
Legend
In the upper panel, we present results from a multivariate linear regression model analysis of actual LTL (mean TRF, dependent variable) on different covariates (rows) in the test data set (comprised of 1078 individuals). The model was regressed on age, gender, race/ethnicity, smoking status, and study cohort. Race/ethnicity is a dichotomized variable (European versus African Ancestry). Smoking status is a three-category variable: never, former and current smokers (as a reference). Study cohort is a trivariate variable (FHS, WHI BA23 and JHS cohort).
In the lower panel, we present the analogous multivariate model but its dependent variable is DNAmTL.

TABLE 3
Overview of the cohorts used in the validation analysis

| | | | | Smoking status | | | Years of |
|---|---|---|---|---|---|---|---|
| Study | N | Female | Age | Never | Former | Current | Follow-up |
| FHS* | 2356 | 54% | 66.4 +/− 8.97 | 9% | 51% | 40% | 7.9 +/− 1.69 |
| | | | [60; 73] | | | | [7.4; 8.9] |
| WHI BA23 | 1389 | 100% | 65.1 +/− 7.25 | 9% | 35% | 54% | 16.6 +/− 4.87 |
| | | | [59.2; 71] | | | | [14.7; 19.9] |
| WHI EMPC | 1972 | 100% | 63.3 ± 7.03 | 52% | 38% | 9% | 18 ± 4.02 |
| | | | [57.9, 68.7] | | | | [17.9, 20.1] |
| JHS | 209 | 56% | 58.2 +/− 12.89 | 13% | 21% | 66% | 11.7 +/− 2.74 |
| | | | [47.1; 69.3] | | | | [11.1; 13.4] |
| InChianti** | 924 | 54% | 67 ± 16.64 | | | | 5.4 ± 4.84 |
| | (484) | | [60, 78] | 57% | 29% | 14% | [0.1, 9.3] |

NA = not available.
Quantitative variables are presented in the format of mean ± SD [25^{th}, 75^{th}].
*The distribution of age is based on exam 8.
**The statistics are based on the number of 924 observations across 484 individuals.
Legend
The table above summarizes the characteristics of individuals from four independent cohorts across five studies that were used in our validation analysis. For example, up to two longitudinal measurements were available for each of the 484 individuals from the InChianti cohort.

TABLE 4
CpG SEQUENCES

| | | SEQ |
|---|---|---|
| cg Identifier | | ID |
| cg05528516 | CGGACCCCCACCGGCCTCCAAATGTGCAAACACAGGCGCCTCTCAGGCAC | 1 |
| cg00060374 | CGGCTTCATGCTGGTGGCAGCAACAGACTCTCCGCCAGCGCCGGGCCTGT | 2 |
| cg12711627 | CGGGCACCACACAGCATCCCAGGCACCATCATGGTAGGAGAAGAGTTCAG | 3 |
| cg06853416 | AGAGTCCCCCTCTGGATTCACACACCTGGAGGCGTCTGAGTGACTCCTCG | 4 |
| cg01901101 | CAAAAAAACCCCAGCTTTTGTCCAGAGGTTGCTTTTTGTGGGTCTGTACG | 5 |
| cg21393163 | AGAAATGCAAATGAAATGACACCGGGCTCCACTTCACACCCATCAGATCG | 6 |
| cg22866430 | GCACCCCCCCATCCACTTCATGTTCAGAAAACTCAAAGAGTCAGAAAACG | 7 |
| cg16047567 | GTCTTCTAAGATTTTCCTTAATTTACAGTGATCAAAGCTCTCAGGCCACG | 8 |
| cg18768612 | CGGGCCTGTATTCAGTCCGCCGTGATGTGAGAACTGAGTATCACTCTTCT | 9 |
| cg24049493 | CGCGGCCACCACAGACCTGAGCCCAGGAGGCTCTGTGACAAGGCACAGAC | 10 |
| cg08893087 | CGGCTTCCAGATTCTTTCCAGTTGTCAGAATTCAGTTACATGTGGTTGCA | 11 |
| cg03984502 | TCTGAGGCTCTGATTATCCCCCGTGTTCTTTTGGTAAATGTGGGCCCACG | 12 |
| cg19233405 | CGGAAGCACACGGGAGAGCGCCCCTACTCATGCCCGCACTGCCCAGCCCG | 13 |
| cg05694771 | CGTGCAAAGTTTGTCTGTCTGTGCCTGGCTTATTTCACGTAACATAATGA | 14 |
| cg24739596 | ACACAGCAGAAGAAGACCCTTGACATCATCATCAGGGTTCTAGATCCACG | 15 |
| cg06370057 | CGCCCAAGCCACCATGAAGGGACGCTCGCACAAAGTGAAGTCCACATACA | 16 |
| cg24457743 | CGGGCTGCAGCTGACATCTTCTTTAAACACTGAGAGTGTGCTGGGGGCAG | 17 |
| cg18148156 | CGGCAGGTTGGAAATCCAGTTTGTGGCTGATGCAAGCAAACCATGCTGCA | 18 |
| cg19935065 | CGGTTGTGAGGTGCTCACGTGTTTTGGAGATAGCAAAAGTCTCAAATAAT | 19 |
| cg10549018 | TGTCTCCTCATCTCCTGGATCTTTGCCCAGCAAAACCTCCAAAGAGACCG | 20 |
| cg24903144 | CGCAAGGTAGAAAGCAAAGGGGGGACTGGAGAGAAACCAGATGACAGAAA | 21 |
| cg17782974 | CGCACACACACACCCTGCGGAGTTGCCGACAAAGTTCAGTGCCTGCTTCC | 22 |
| cg13357922 | CGCTACCACTAGACCAAGGTCAGCCAAGTAACAGTTGTCTTCCCAGACGC | 23 |
| cg23908305 | CGGCCCTTGTTAGGGACTGGCTACAGCGGCTGGCCCCAAGGGCCCCTGTG | 24 |
| cg15742496 | CGGGCACACAGCCAGTGTCTGTCAGAGTCAGGATTTGAACACATGCAGTC | 25 |
| cg27639942 | ATAGCCTGGCCGAGCCTGGGAACCACCTGGGGCTGAGGTAACGGCACACG | 26 |
| cg27312916 | CGACAGGTGCAGAGAGAGAATAAACTGTGTCTAAGACTGGACCAGAGAGT | 27 |
| cg02121547 | CAGCCTGGGGACAAGAACATCAAATGTCCGAGTCTGAGCTGCCTGGTTCG | 28 |
| cg26827653 | CGGAAAGTCCAAAAGGGTCTTTGAGTGTGTCTGCCGGCATCCGCCAGCAG | 29 |
| cg16593899 | TAAAGCCCTGTCATTAGCTGGCAGCAGGGAGGCGGTCGGTAGGGGCAACG | 30 |
| cg02319782 | CGGGTTCCCTCCCTAGGGAACCAGGCTGCCTAACCCACCAGCCGGCCCCT | 31 |
| cg26276120 | CGGCATAGGCACCTCACAGACTCAGCATTCTCCAGGGAGCAACCCTGCTT | 32 |
| cg12745325 | CTCCTCTGATGTCTGACCTGGCCTAAAATCAGGCAAGCAAAGTCTGATCG | 33 |
| cg07677157 | CGGTGATCCATAGCAAAAGAGCCTATGAGTCAGGTGATGATCCATTCATC | 34 |
| cg07910460 | CGCGCTCCTCCACTGTCTGAACCAAGTTAGGAGCCCTGATTAAACCTCCG | 35 |
| cg01614102 | CAAAGGAAATCGCAGGCGTCTTTGCAAAGCTGGTGGGAAGTAACCCAGCG | 36 |
| cg20742385 | CGGCTGGGTTTCTCTAGCACCTCGGCATTGTCTGCCTGCTCTTCCAGGCG | 37 |
| cg05019423 | CGGGTGTCAAAGTCAGTGGATGGCAAAACATGCCTGGAGGGAGCAACCAG | 38 |
| cg19825410 | TCTCACCAACTCTCTTGTCCACCTTGGTGTTGCTGGGCTTGTGATCTACG | 39 |
| cg02963381 | CGGCCCCCAGCGCCCGCGCCCATCCTGGAGAACTGCATCTGCGCAGGCCC | 40 |
| cg00843787 | GATTTGTATTCTTTTCTTTTCCTTTTTTGAGATGGAGTCTAGCTCTGTCG | 41 |
| cg08107689 | CGCGGCGTCGCCCTCCGAGTCGCGCTCAGAGCCACTGCTTTATGTTCAAA | 42 |
| cg04875128 | CGCCACGTACCCGCAGCAGAACCGCTCGCTGTCGTCGCAGAGCTACAGCC | 43 |
| cg26709300 | CGGTGACAATCATCCAAGTAGGCCTGAAACGTCTTGGGCTTTGAAATCCG | 44 |
| cg27093918 | CGCACTAAGAAGGGCTCCTGGGTGCTCAGCACCTAAAGTGGGTCATTCAT | 45 |
| cg02282640 | ACTTTAGATCAGGTCAAAGGTTCCAATTACGAAGCTAAAAGAATTCGGCG | 46 |
| cg02380595 | CGGAGAGGGGCGCCTTCTCCATCTGTACGGCCTCACCGGGACTTTCCTCC | 47 |
| cg04658841 | CGGGGGCTGTCTTTTCTGCACGCCTTGCCCTTGATCTTCCCTGGCTTTAT | 48 |
| cg07955474 | CAACATTTCCAAGAACAGTGCTGGAAATCCCCAAGACAGCTGGATAGACG | 49 |
| cg07708487 | GGAGTGTGCCAGTGGCTCGTGTGGGGGACTCCCACCACCCTGGACTCTCG | 50 |
| cg00974523 | GGCTTCTCCCCTGTGTGTGTCCTCTGGTGACTGAGGAGGTCTGACTTCCG | 51 |
| cg15825321 | CGGCCGTCCTTCAGCTCGCACCACCTCATGCCCTGCGACACTCTGGCGCG | 52 |
| cg14244013 | TCACAGGCTCTGGGTTCGCGTTCGGAAACGCTTTCGCAGGGAGCGGGTCG | 53 |
| cg25583580 | CGTATTCCTGGCCAAATAACCAAGGTGAAGAAAGGTAAGGGGAAGAAAAA | 54 |
| cg18826274 | CGCCCTGAAAGATTGTAGTACAGAGTCCTGGGGAGAAAGTGGTTTCCTGC | 55 |
| cg18836174 | CTCCAACCCCAGCTTTTTCACTAGTAAGGCAGTCGGGCCCCTGGGCCACG | 56 |
| cg06994022 | AGATTGTGTTTTTCCTTTCTAAAGCCTCCTGCTTCATAGTAACTTCCACG | 57 |
| cg19283806 | GATTTCTCCTTGAACAATCCCCGCAAAGATAGCAGCCAAAAAAGGATGCG | 58 |
| cg23686403 | TGGCCGCGATGCTCCTTTCCCCAGGCTCTGGGATTGTGGACAGGAGGGCG | 59 |
| cg02194396 | ACATCTGTGTTGAGCTTTTTTTAGATCTTCCCAAACAATCTGGCATTTCG | 60 |
| cg07069844 | CGTGGAAGTCCGCTTCAGCACATCAAGAGATTAAGGAAATGCAAATTAGG | 61 |
| cg00593900 | CGCGAGCTGGCGCAGCTCGTCACCCAGCAGAGCAGTCTCATCGCCCGCCT | 62 |
| cg13523818 | CGGCAGGGCAAGACGATGGTGCCACCACGGTGGCTTACCACCTGCCCAGG | 63 |
| cg26950531 | TGCCGGCCACGTGGTTGCTGGGCAGGTGTGGGGTCAGGCTGTGGATCCCG | 64 |
| cg06673536 | CGCTGAGCTGTTGGGGAGCCACCGAGGCCATAAACGTCCTGGTTAATGCA | 65 |
| cg19327213 | CGAGGTGTCCAGGACAGCTGGCCAACCCATGTCTTCTGGAAATGATACAC | 66 |
| cg26636010 | GGTGAGCGCCGGCTGGGCTTGATGGGAGGCTGAATCCCGTTCGTGGACCG | 67 |
| cg10550416 | TTCTGATGTGCTCGTCTGGTAGTTCTAAGGTGGTTATGGCAGCTGGGTCG | 68 |
| cg01603921 | CGGCTTTTTGTGCCCATCACCGTAGCTTTTTGCCTCCGCCACCACAGCTT | 69 |
| cg23631636 | TGGGCTCGGACCTAGGTCGCGGCGACATGGTGAGTGTGGGTCTCTGTGCG | 70 |
| cg07374224 | GGGCAGGTTCCTTGGTGACAAAGCCATGAGCATGGCCAGCAGCCCTTGCG | 71 |
| cg06139893 | CTGTGTGTCAGCCACGGCCAGGGTGGGGCAGCCTGTCCATCTGGGTGACG | 72 |
| cg09374293 | CGATTCTGTCATGAAGAGTGGCTTAAAATACTTGAAAAAAACTGTAATGC | 73 |
| cg21461082 | CGCTGAATGCGTCAGGAACCGTAGAAAAGCCCCCCAGCCTGGGGAAACCA | 74 |
| cg01239389 | AGCCGCAGCGGCGGGGGGCAAAAAGCCGTGGCTGGCAAAAAGCCGAGGCG | 75 |
| cg06845706 | CGGCCCCATGAGTGCTCTTCGGCCGCCCAAAACCGGTCCCATGGGTAACA | 76 |
| cg15223899 | CGGCACGTGCCAGGACACGTTCCTCTTGCCGCCTCCGCAGCACACGGCCA | 77 |
| cg00733150 | CGCGCGCCCCGCCCCGCCCCGCCGCCAGATCCTCTGGCACCGCCCACCTC | 78 |
| cg18405719 | CGAGGCTAAAATGTGGGGCCAGAAGATAAATCTAATGAGAGCTGTTGCTA | 79 |
| cg04508804 | CGCCCCGGTCTGCTGCTAAGCACAGCACAGTTACCAAAGCCAGGAAACTA | 80 |
| cg00840990 | CCACATTACTAGGTAATAAGGTTGCAAATTCCAAGTCTCCCTTGTAGTCG | 81 |
| cg09217898 | CGTGTGTGGACACACATGGTTTCCCTAAAGAAACACCTCTCACTGCCCCA | 82 |
| cg05023043 | CGCCCCTGGTTTGTAGGCAGGCGTTCAGAAATGAAGACGGGTGTCCAGAG | 83 |
| cg19651128 | CGGCCAGGCAAGCCGGCACCGCCCCCGCCATCAGACACCATCTCCCGCCC | 84 |
| cg02810967 | CGTACTGGTACTAAAGGCTTATTGAGAATCAAAGATATTGAAAAAAATAT | 85 |
| cg09435170 | CGGGAAAATCACATCTGTTCTTACCAGTTCATTCTTGGTACTATGACTCA | 86 |
| cg00580497 | CGGCTTGTGGCAACAGGGGCTTCCCCATGGGCTGTTCCTTGGCTCTGTAG | 87 |
| cg00461022 | CGGAGTGGCTCTGAAATTACTGTCTTCAAAGCAGGCCAAACCTTGCTTGA | 88 |
| cg22657457 | CTACAAAAATTCCTTGAACAAATTGTACAGTCAAAGCAGACTTTCCAACG | 89 |
| cg10616795 | GGAGGCCAGGGAGCAGCGAGGCTGCACGGCAGCCCTGGCACAGAGCCTCG | 90 |
| cg06677021 | CGCTGCCTGGGGCCCTCAGCAGCGATCTTAGCTTAGTTACATTTCCCATC | 91 |
| cg02403883 | CGGGTTGTTTTCAAAGGGAACAGACAACATTTGCAGTTTGATCCACAGAC | 92 |
| cg21777188 | CGCCCAGAACTGGGACTTCCCAGGTTCTCATAGTGATTGTAGATATCAAT | 93 |
| cg03335262 | GAAAACCGAGTACAACATAACCCTGCGGGTCTCCGACGTCAATGACAACG | 94 |
| cg27582059 | TCTGGTGGACGTGAGGGGCACCGGGAGCCTGTCTCAGAACTATCAGTACG | 95 |
| cg08985570 | CCAGCACGGTTATGTTGTGCTCGGTTTTTAGCCTGGGGGTCCCCAAGTCG | 96 |
| cg27577149 | CGCCTCCCGAAGCCTGTAGCAGACGTGACTTGTGCCAGCGCCGCAGCCTG | 97 |
| cg13650304 | CCTTGGTCACCAGGTAGCCAGGTTCTGCGGAGCGAGGCGCCAGCTCCACG | 98 |
| cg19247475 | CGCTTCATCTGGTCTTCGCAGACAACTTGCAAGAGATACTGCCAGACCTC | 99 |
| cg10679597 | ATACTTCTAAGTCTCCAGTGTCAGGATCTGTGAGTATCCATCTCATTCCG | 100 |
| cg18104870 | CGGCTTGGGCCCTCCTTGTGGCTCATTGCACCCAGGAGGCAGTTCAGCGG | 101 |
| cg16867657 | CGGCGGCTCAACGTCCACGGAGCCCCAGGAATACCCACCCGCTGCCCAGA | 102 |
| cg22328256 | ACCCAATGGGTTGCCTCCATTTATAACCCATTTCTATGCCGTGAGCTTCG | 103 |
| cg17636541 | CGGCCGAGAAGCTACAGGGGAGGGAGACCTGAGTTCTAAATTAGGAACTC | 104 |
| cg01517384 | TCAGGAAAACTCATGCCATTCTCCATTCAACGGAGGGCGACATTCTAGCG | 105 |
| cg27014438 | CGGGATTCAGAGCTAGGTAATCCACAAGAGGAAACCCACCTTAAAGGAAA | 106 |
| cg07739478 | CGGTGGGGTAGAGTCTAAGTTGTTTCCGGTCTGAGCCTTCAAGTTGCTGG | 107 |
| cg08453194 | TCCTCCTCTCCAAGATTATACCTTTGCCATGTACCCGCCATCCATGATCG | 108 |
| cg21288889 | AACAACAAAAAACTTAAATTTCCCTTGACTTTTAAGTTCACTCTTGTTCG | 109 |
| cg17885226 | GAGTCTGTCACTTCCTGAGGGAGCGCGGAGGTCGAGAGACCTGTGAGGCG | 110 |
| cg06638568 | GGCTGAAGCGGCAGCATCGCACTCTCAAAGGGAACAGCGAGACCACACCG | 111 |
| cg20978460 | CGCCGAGGAGAGAGCTGCCACGGCAGCAGGGAACGTGACTCCCTTCACTG | 112 |
| cg03366574 | CCGCCAGGACAGAGAAGCCGGTACCCCAGCGGGTGGTCTGCTTTTCTGCG | 113 |
| cg10536999 | CGACTCTCCCGGCCTAACGCAAGGACCTTCGCCCTGTTTTCAGCCCAATT | 114 |
| cg08972170 | ACTTCACACTGAGTGTAGTGACTGTGTGTCTTTGTTTTCTCCTTCCGTCG | 115 |
| cg06132400 | CGGCAGCGGTTCTGTCGCGGGGACAAATGCCCTTTATAGATATTTTCTAA | 116 |
| cg07076816 | CGGCTACCCCTGCGGCCATGGACGCCATCTAAGCCATGAGGCTCGGCTGG | 117 |
| cg00739278 | GGGGCTGGGGGGCAGCGCGTGTGGGGGTCTGCTGGGCAGCAGGCTGGACG | 118 |
| cg10691866 | CGGTAAACACAGAGCAGGAGGGAATTACAGTGAATGGGGATTTCCCTCAG | 119 |
| cg00277397 | CGCTGTGTGTGTTGAGACACACGCAACATTCGTGCACATAGAACTCATGA | 120 |
| cg14989226 | GGGAGAGCTGCCCCTCCCCAGACCTCAGGTCTCAGCAGCAAACATGTACG | 121 |
| cg09596818 | CGGGCACTGTTGGACCACGTGGCTCCATCATGATGACTCCAGTTAGATGT | 122 |
| cg22633390 | GCCGGCTAAGCCGCGGCGGACAACTATGCTGAAAGCCAAGATCCTCTTCG | 123 |
| cg03473532 | CGTATGTGTTTGAGATAGCAGTTGTTTACTATCACTTGAAAATTCTGAAT | 124 |
| cg00029246 | ACCTTCACCAGGATAAGATCTCCCCTGTTTTTTGAAGCAGAGAACTATCG | 125 |
| cg22530232 | CGGTGTGGGACTAATGGGGACTAATTTATTTAGCTCGATTCCATTTGTCT | 126 |
| cg12133423 | TGTCTGTCTCCATCCTTTCGACCACCTCCAGAAGCTACAGGAAATAAACG | 127 |
| cg16479633 | GTCACTAATGGGTCACAGAGCTTTGTATCCTGAGCTAGTGGCAAAACTCG | 128 |
| cg18898125 | CGCCTTACCTTCTGCCCCTGCGGAAGCTGCGGACGTCCCCCAAACGTTAA | 129 |
| cg16886403 | CGGAGTCCCCAGCAGTGTACAGCGTCTGTTATAAAGTCCCCCAAAGTGTA | 130 |
| cg07211259 | ACAAACTGCTAAAAGCAAAACCAAAACTTTCCAAATAAGCCAGGCTTTCG | 131 |
| cg14577706 | CGCAGGTTGACAAAATCGGCAGATAAAAATACAAGACTGCCATTAAATTT | 132 |
| cg00232500 | ATGCATGCACACATAGTGACCCACCCCACACATGTGCATGCATTCTCACG | 133 |
| cg05072215 | CGCCTGGCCAGACTGTAATTTAAATAAGGGTTAAACTATGTGACAATACA | 134 |
| cg13753488 | TGCTCTCCAGGGGCTTGTGGTTGTAGCCCCCAAGGAAAGGCAGGTGCACG | 135 |
| cg13764516 | CGGGCGCTCCGAACCTTGTGGTCTGAGGCAGGAACTCCATTAAATCAGCT | 136 |
| cg07096038 | CGTGGAGGCACCGGGAGGCGTGTGGGGGGCGGTCGGCGAGCGGAGATCCG | 137 |
| cg16203368 | CGGGGGTCCCCCAATTCCAGAAGAACTGGAAAAGCACATGGGGACCCCCT | 138 |
| cg00780578 | CATCCAGCATGGCCCTTGTGGACTTCTGCAGGGCTTCCATGCTTTTAGCG | 139 |
| cg16708895 | CCACTAGCTGGAGAGGTAATTTTGCGTCTAATTTTGCACCTCTCTCCTCG | 140 |

### REFERENCE LIST

The following references are cited in, and pertain to, the disclosure immediately above this section except for the references listed in the background and summary disclosure.
1 Nordfjäll, K., Svenson, U., Norrback, K.-F., Adolfsson, R. & Roos, G. Large-scale parent-child comparison confirms a strong paternal influence on telomere length. European journal of human genetics: EJHG 18, 385-389, doi:10.1038/ejhg.2009.178 (2010).
2 Daniali, L. et al. Telomeres shorten at equivalent rates in somatic tissues of adults. Nat Commun 4, 1597, doi:10.1038/ncomms2602 (2013).
3 Epel, E. S. et al. Accelerated telomere shortening in response to life stress. Proc Natl Acad Sci USA 101, 17312-17315, doi:10.1073/pnas.0407162101 (2004).
4 Fitzpatrick, A. L. et al. Leukocyte Telomere Length and Cardiovascular Disease in the Cardiovascular Health Study. American Journal of Epidemiology 165, 14-21, doi:10.1093/aje/kwj346 (2007).
5 Willeit, P. et al. Cellular aging reflected by leukocyte telomere length predicts advanced atherosclerosis and cardiovascular disease risk. Arteriosclerosis, thrombosis, and vascular biology 30, 1649-1656 (2010).
6 Mather, K. A., Jorm, A. F., Parslow, R. A. & Christensen, H. Is Telomere Length a Biomarker of Aging? A Review. The Journals of Gerontology Series A: Biological Sciences and Medical Sciences 66A, 202-213, doi:10.1093/gerona/glq180 (2011).
7 Needham, B. L. et al. Leukocyte telomere length and mortality in the National Health and Nutrition Examination Survey, 1999-2002. Epidemiology 26, 528-535, doi:10.1097/EDE.0000000000000299 (2015).
8 Chen, B. H. et al. Leukocyte telomere length, T cell composition and DNA methylation age. Aging 9, 1983-1995, doi:10.18632/aging.101293 (2017).
9 Jylhava, J., Pedersen, N. L. & Hagg, S. Biological Age Predictors. EBioMedicine 21, 29-36, doi:10.1016/j.ebiom.2017.03.046 (2017).
10 Aviv, A. & Shay, J. W. Reflections on telomere dynamics and ageing-related diseases in humans. Philos Trans R Soc Lond B Biol Sci 373, doi:10.1098/rstb.2016.0436 (2018).
11 Hannum, G. et al. Genome-wide methylation profiles reveal quantitative views of human aging rates. Molecular cell 49, 359-367, doi:10.1016/j.molcel.2012.10.016 (2013).
12 Horvath, S. DNA methylation age of human tissues and cell types. Genome biology 14, R115, doi:10.1186/gb-2013-14-10-r115 (2013).
13 Lin, Q. et al. DNA methylation levels at individual age-associated CpG sites can be indicative for life expectancy. Aging 8, 394-401 (2016).
14 Horvath, S. et al. Epigenetic clock for skin and blood cells applied to Hutchinson Gilford Progeria Syndrome and ex vivo studies. Aging 10, 1758-1775, doi:10.18632/aging.101508 (2018).
15 Levine, M. E. et al. An epigenetic biomarker of aging for lifespan and healthspan. Aging, doi:10.18632/aging.101414 (2018).
16 Horvath, S. & Raj, K. DNA methylation-based biomarkers and the epigenetic clock theory of ageing. Nat Rev Genet, doi:10.1038/s41576-018-0004-3 (2018).
17 Lu, A. T., Quach, A., Ferrucci, L., Assimes, T. & Horvath, S. DNA methylation GrimAge strongly predicts lifespan and healthspan. accepted by Aging (2019).
18 Chen, B. et al. Leukocyte Telomere Length and DNA Methylation Age. (2017).
19 Lu, A. T. et al. GWAS of epigenetic aging rates in blood reveals a critical role for TERT. Nat Commun 9, 387, doi:10.1038/s41467-017-02697-5 (2018).
20 Marioni, R. E. et al. The epigenetic clock and telomere length are independently associated with chronological age and mortality. International Journal of Epidemiology 45, 424-432, doi:10.1093/ije/dyw041 (2016).
21 The, B. c. et al. Quantitative comparison of DNA methylation assays for biomarker development and clinical applications. Nature Biotechnology 34, 726, doi:10.1038/nbt.3605 https://www.nature.com/articles/nbt.365#supplementary-information (2016).
22 Montpetit, A. J. et al. Telomere Length: A Review of Methods for Measurement. Nursing research 63, 289-299, doi:10.1097/NNR.0000000000000037 (2014).
23 Denham, J., Marques, F. Z. & Charchar, F. J. Leukocyte telomere length variation due to DNA extraction method. BMC Res Notes 7, 877, doi:10.1186/1756-0500-7-877 (2014).
24 Kimura, M. et al. Measurement of telomere length by the Southern blot analysis of terminal restriction fragment lengths. Nat Protoc 5, 1596-1607, doi:10.1038/nprot.2010.124 (2010).
25 Grundberg, E. et al. Global Analysis of DNA Methylation Variation in Adipose Tissue from Twins Reveals Links to Disease-Associated Variants in Distal Regulatory Elements. The American Journal of Human Genetics 93, 876-890, doi:10.1016/j.ajhg.2013.10.004 (2013).
26 Horvath, S. et al. Obesity accelerates epigenetic aging of human liver. Proceedings of the National Academy of Sciences of the United States of America 111, 15538-15543, doi:10.1073/pnas.1412759111 (2014).
27 Ahrens, M. et al. DNA Methylation Analysis in Nonalcoholic Fatty Liver Disease Suggests Distinct Disease-Specific and Remodeling Signatures after Bariatric Surgery. Cell Metabolism 18, 296-302, doi:10.1016/j.cmet.2013.07.004 (2013).
28 Reynolds, L. M. et al. Age-related variations in the methylome associated with gene expression in human monocytes and T cells. Nat Commun 5, 5366, doi:10.1038/ncomms6366 (2014).
29 Zou, H. & Hastie, T. Regularization and variable selection via the elastic net. Journal of the Royal Statistical Society: Series B (Statistical Methodology) 67, 301-320, doi:10.1111/j.1467-9868.2005.00503.x (2005).
30 Dalgard, C. et al. Leukocyte telomere length dynamics in women and men: menopause vs age effects. Int J Epidemiol 44, 1688-1695, doi:10.1093/ije/dyv165 (2015).
31 Hunt, S. C. et al. Leukocyte telomeres are longer in African Americans than in whites: the National Heart, Lung, and Blood Institute Family Heart Study and the Bogalusa Heart Study. Aging cell 7, 451-458, doi:10.1111/j.1474-9726.2008.00397.x (2008).
32 Levine, M. E. et al. Menopause accelerates biological aging. Proceedings of the National Academy of Sciences of the United States of America 113, 9327-9332, doi:10.1073/pnas.1604558113 (2016).
33 Zheng, Y. L. et al. Telomerase enzymatic component hTERT shortens long telomeres in human cells. Cell Cycle 13, 1765-1776, doi:10.4161/cc.28705 (2014).
34 Horvath, S. et al. An epigenetic clock analysis of race/ethnicity, sex, and coronary heart disease. Genome biology 17, 171, doi:10.1186/s13059-016-1030-0 (2016).
35 Houseman, E. et al. DNA methylation arrays as surrogate measures of cell mixture distribution. BMC bioinformatics 13, 86 (2012).
36 McLean, C. Y. et al. GREAT improves functional interpretation of cis-regulatory regions. Nat Biotechnol 28, doi:10.1038/nbt.1630 (2010).
37 Horvath, S. DNA methylation age of human tissues and cell types. Genome Biol 14, doi:DOI: 10.1186/10.1186/gb-2013-14-10-r115 (2013).
38 Chen, W. et al. Longitudinal versus cross-sectional evaluations of leukocyte telomere length dynamics: age-dependent telomere shortening is the rule. The journals of gerontology. Series A, Biological sciences and medical sciences 66, 312-319, doi:10.1093/gerona/glq223 (2011).
39 Steenstrup, T., Hjelmborg, J. V., Kark, J. D., Christensen, K. & Aviv, A. The telomere lengthening conundrum—artifact or biology? Nucleic acids research 41, e131, doi:10.1093/nar/gkt370 (2013).
40 Kabacik, S., Horvath, S., Cohen, H. & Raj, K. Epigenetic ageing is distinct from senescence-mediated ageing and is not prevented by telomerase expression. Aging 10, 2800-2815, doi:10.18632/aging.101588 (2018).
41 Vetter, V. M. et al. Epigenetic Clock and Relative Telomere Length Represent Largely Different Aspects of Aging in the Berlin Aging Study II (BASE-II). The journals of gerontology. Series A, Biological sciences and medical sciences 74, 27-32, doi:10.1093/gerona/gly184 (2019).
42 Rizos, E. C., Ntzani, E. E., Bika, E., Kostapanos, M. S. & Elisaf, M. S. Association between omega-3 fatty acid supplementation and risk of major cardiovascular disease events: a systematic review and meta-analysis. JAMA 308, 1024-1033, doi:10.1001/2012.jama.11374 (2012).
43 Aung, T. et al. Associations of Omega-3 Fatty Acid Supplement Use With Cardiovascular Disease Risks: Meta-analysis of 10 Trials Involving 77917 Individuals. JAMA Cardiol 3, 225-234, doi:10.1001/jamacardio.2017.5205 (2018).
44 Rizos, E. C. & Elisaf, M. S. Does Supplementation with Omega-3 PUFAs Add to the Prevention of Cardiovascular Disease? Curr Cardiol Rep 19, 47, doi:10.1007/s11886-017-0856-8 (2017).
45 Dawber, T. R., Meadors, G. F. & Moore, F. E., Jr. Epidemiological approaches to heart disease: the Framingham Study. Am J Public Health Nations Health 41, 279-281 (1951).
46 Anonymous, A. Design of the Women's Health Initiative clinical trial and observational study. The Women's Health Initiative Study Group. Control Clin Trials 19, 61-109 (1998).
47 Anderson, G. et al. Implementation of the women's health initiative study design. Ann Epidemiol 13, S5-17 (2003).
48 Taylor, H. A., Jr. et al. Toward resolution of cardiovascular health disparities in African Americans: design and methods of the Jackson Heart Study. Ethn Dis 15, S6-4-17 (2005).
49 Triche, T. J., Weisenberger, D. J., Van Den Berg, D., Laird, P. W. & Siegmund, K. D. Low-level processing of Illumina Infinium DNA Methylation Bead Arrays. Nucleic acids research 41, e90-e90, doi:10.1093/nar/gkt090 (2013).
50 Fortin, J.-P., Triche, T. J., Jr. & Hansen, K. D. Preprocessing, normalization and integration of the Illumina Human Methylation EPIC array with minfi. Bioinformatics (Oxford, England) 33, 558-560, doi:10.1093/bioinformatics/btw691 (2017).
51 Lu, A. T. et al. Genetic architecture of epigenetic and neuronal ageing rates in human brain regions. Nat Commun 8, 15353, doi:10.1038/ncomms15353 (2017).
52 Lu, A. T. et al. Genetic variants near MLST8 and DHX57 affect the epigenetic age of the cerebellum. Nat Commun 7, 10561, doi:10.1038/ncomms10561 (2016).
53 Almasy, L. & Blangero, J. Multipoint quantitative-trait linkage analysis in general pedigrees. American journal of human genetics 62, 1198-1211, doi:10.1086/301844 (1998).
54 Ziyatdinov, A. et al. solarius: an R interface to SOLAR for variance component analysis in pedigrees. Bioinformatics 32, 1901-1902, doi:10.1093/bioinformatics/btw080 (2016).
55 Horvath, S. & Levine, A. J. HIV-1 infection accelerates age according to the epigenetic clock. J Infect Dis, doi:10.1093/infdis/jiv277 (2015).
56 Langfelder, P. & Horvath, S. WGCNA: an R package for weighted correlation network analysis. BMC Bioinformatics 9, 559 (2008).
57 Quach, A. et al. Epigenetic clock analysis of diet, exercise, education, and lifestyle factors. Aging (Albany N.Y.), doi:10.18632/aging.101168 (2017).

All publications mentioned herein (e.g. those cited above) are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification.

### CONCLUSION

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A method of obtaining information on mean telomere length in an individual, the method comprising:
observing methylation of methylation markers present in SEQ ID NO.:1-SEQ ID NO.: 140 in genomic DNA from the individual; and
correlating methylation observed with mean telomere length;
such that information on mean telomere length in the individual is obtained.

2. The method of claim 1, wherein genomic DNA used in the method is obtained from human leukocytes, fibroblasts, keratinocytes, buccal cells, endothelial cells, lymphoblastoid cells, and/or cells obtained from human blood, skin or saliva.

3. The method of claim 1, further comprising using the observations to estimate the phenotypic age of the individual.

4. The method of claim 3, further comprising comparing the estimated phenotypic age with the actual age of the individual so as to obtain information on life expectancy or time-to-heart disease of the individual.

5. The method of claim 1, further comprising using the observations to estimate the abundance of CD8+ T cells in the individual.

6. The method of claim 1, wherein correlating methylation observed with mean telomere length comprises use of a weighted average of methylation markers.

7. The method of claim 1, wherein correlating methylation observed with mean telomere length comprises use of a regression analysis.

8. The method of claim 1, wherein:
methylation is observed by a process comprising treatment of genomic DNA from the population of cells from the mammals with bisulfite to transform unmethylated cytosines of CpG dinucleotides in the genomic DNA to uracil; and/or
methylation is observed by a process comprising hybridizing genomic DNA obtained from the individual with 140 complementary sequences disposed in an array and coupled to a substrate.

9. The method of claim 1, wherein information obtained in the method is used to determine mean telomere length in the individual.

10. A method of observing effects of a test agent on genomic methylation associated epigenetic aging of human cells, the method comprising:
(a) combining the test agent with human cells;
(b) observing methylation in methylation markers of SEQ ID NO.:1-SEQ ID NO.: 140 in genomic DNA of the human cells in (a); and
(c) comparing the observations from (b) with observations of the methylation status in genomic DNA from control human cells not exposed to the test agent such that effects of the test agent on genomic methylation associated epigenetic aging in the human cells are observed.

11. The method of claim 10, wherein genomic DNA used in the method is obtained from human leukocytes, fibroblasts, keratinocytes, buccal cells, endothelial cells, lymphoblastoid cells, and/or cells obtained from human blood, skin or saliva.

12. The method of claim 10, wherein a plurality of test agents are combined with the mammalian cells.

13. The method of claim 10, wherein the test agent is a compound having a molecular weight less than 3,000 g/mol.

14. The method of claim 10, wherein the test agent is a polypeptide or a polynucleotide.

15. The method of claim 10, wherein the method further comprises comparing methylation profiles in cells from different tissue lineages.

16. The method of claim 10, wherein genomic DNA used in the method is obtained from human cells of a leukocyte lineage, a neural cell lineage, a cardiac cell lineage or a skin cell lineage.

17. The method of claim 10, wherein:
methylation is observed using a weighted average of methylation markers; and/or
methylation is observed using a regression analysis.

18. The method of claim 10, wherein methylation is observed by a process comprising treatment of genomic DNA from the population of cells from the mammals with bisulfite to transform unmethylated cytosines of CpG dinucleotides in the genomic DNA to uracil.

19. The method of claim 10, wherein genomic DNA is amplified by a polymerase chain reaction process.

20. A tangible computer-readable medium comprising computer-readable code that, when executed by a computer, causes the computer to perform operations comprising:
a) receiving information corresponding to methylation levels of a set of methylation markers in a biological sample, wherein the set of methylation markers comprises methylation markers in SEQ ID NO.:1-SEQ ID NO.: 140;
b) characterizing telomere length by applying an algorithm to methylation data obtained from the set of methylation markers; and
c) determining mean telomere length.
